(19) United States
(12) Patent Application Publication
(10) Pub. No.: US 2024/0366815 A1
(43) Pub. Date: Nov. 7, 2024

(54) TREATMENT OF PROSTATE CANCER WITH RADIOLABELED ANTIBODIES TARGETING CDCP1

(71) Applicant: The Regents of the University of California, Oakland, CA (US)

(72) Inventors: Michael J. EVANS, Pacifica, CA (US); James A. WELLS, Burlingame, CA (US); Rahul AGGARWAL, Orinda, CA (US)

(73) Assignee: The Regents of the University of California, Oakland, CA (US)

(21) Appl. No.: 18/655,103

(22) Filed: May 3, 2024

Related U.S. Application Data
(60) Provisional application No. 63/500,542, filed on May 5, 2023.

(51) Int. Cl. A61K 51/10; A61K 9/00; A61P 35/00; C07K 16/28

(52) U.S. Cl. CPC A61K 51/1027; A61K 9/0019; A61P 35/00; C07K 16/2896; A61K 2121/00; A61K 2123/00; C07K 2317/565

(57) ABSTRACT

The disclosure relates to radioimmunotherapy for prostate cancer using an antibody or antibody fragment that specifically binds CUB domain-containing protein 1 (CDCP1). The disclosure further related to use of a diagnostic radioisotope-labeled anti-CDCP1 antibody or CDCP1-binding fragment thereof for radionuclide imaging of CDCP1 expression in vivo. in prostate cancer patients.

### CROSS-REFERENCE TO RELATED APPLICATIONS

This application claims priority to and benefit of U.S. Provisional Patent Application No. 63/500,542, filed May 5, 2023, the content of which is herein incorporated by reference in its entirety.

### STATEMENT REGARDING FEDERALLY SPONSORED RESEARCH OR DEVELOPMENT

This invention was made with government support under W81XWH-21-1-0498 awarded by the Medical Research and Development Command. The government has certain rights in the invention.

### REFERENCE TO AN ELECTRONIC SEQUENCE LISTING

The content of the electronic sequence listing (64/366,2003100SEQLIST.xml; Size: 73,085 bytes; and Date of Creation: Apr. 18, 2024) is herein incorporated by reference in its entirety.

### FIELD

The disclosure relates to radioimmunotherapy for prostate cancer using an antibody or antibody fragment that specifically binds CUB domain-containing protein 1 (CDCP1). The disclosure also relates to use of a diagnostic radioisotope-labeled anti-CDCP1 antibody or CDCP1-binding fragment thereof for radionuclide imaging of CDCP1 expression in vivo in prostate cancer patients.

### BACKGROUND

In the United States, one in eight men will be diagnosed with prostate cancer during his lifetime, and one in 41 men will die of prostate cancer (American Cancer Society). Globally, prostate cancer is one of the foremost causes of cancer-related morbidity and mortality in men. Treatment options and prognosis are dependent on the stage of the prostate cancer.

Androgen deprivation therapy (ADT) is an antihormone therapy, which involves reducing levels of androgen hormones through surgical castration through orchiectomy, or chemical castration through administration of lutenizing hormone receptor hormone analogs or antiandrogen agents. ADT is an important component of first line treatment for locally advanced and metastatic prostate cancer. However, effectiveness of ADT is temporary as prostate cancer cells frequently develop resistance within two or three years even when testosterone levels remain below the castration level (Yeha et al., Cancer Drug Resist, 5:667-90, 2022). Castration-resistant prostate cancer (CRPC) can progress to metastatic CPRC (mCPRC), which has a mean overall survival of less than two years (Yeha et al, supra, 2022).

The Food and Drug Administration (FDA) has recently approved PLUVICTO® (Lutetium Lu-177 Vipivotide Tetraxetan, marketed by Novartis AG) as a radioligand therapeutic agent for treatment of patients with prostate-specific membrane antigen (PMSA)-positive mCRPC who have previously been treated with androgen receptor pathway inhibition and taxane-based chemotherapy (Hennrich et al., Pharmaceuticals, 15:1292, 2022). Importantly, PSMA-directed radioligand therapy (RLT) is not suitable for treating low-PSMA expressing mCRPC, including treatment-emergent small cell neuroendocrine cancer (t-SCNC), a subset of mCRPC observed in up to 15-20% of cases (Aggarwal et al., J Natl Compr Canc Netw, 12:719-726, 2014). PSMA is also not selectively expressed by mCRPC, and on-target, off-tissue effects of PSMA-directed RLT (e.g., xerostomia) are well documented, limiting treatment duration and overall quality of life.

Thus, the identification of additional cell surface antigens as targets for treatment of mCRPC are needed, particularly for low-PSMA expressing mCRPC.

### SUMMARY

The disclosure relates to radioimmunotherapy for prostate cancer using an antibody or antibody fragment that specifically binds CUB domain-containing protein 1 (CDCP1). The disclosure further related to use of a diagnostic radioisotope-labeled anti-CDCP1 antibody or CDCP1-binding fragment thereof for radionuclide imaging of CDCP1 expression in vivo in prostate cancer patients.

### BRIEF DESCRIPTION OF THE DRAWINGS

FIG. 1A-1C show expression of CDCP1 in metastatic castration-resistant prostate cancer (mCRPC). FIG. 1A are histograms showing the distribution of CDCP1 mRNA levels over the set of 119 mCRPC biopsies, with the distribution of FOLH1 (PSMA) mRNA levels shown for comparison. FIG. 1B is a scatter plot showing the expression of CDPC1 and FOLH1 per biopsy. FIG. 1C shows the mean H-scores for CDCP1 membrane staining from 17 evaluable mCRPC biopsies. The H-scores were determined by two blinded pathologists and the mean reported. Four of seventeen biopsies (23%) had higher CDCP1 H-scores compared to PSMA. All biopsies were histologically adenocarcinoma.

FIG. 2A-2D show that CDCP1 is abundantly expressed in human prostate cancer models, including prostate-specific membrane antigen (PSMA) low adenocarcinoma and small-cell neuroendocrine carcinoma (SCNC). FIG. 2A is an immunoblot showing CDCP1 expression in whole cell lysates of patient-derived xenografts (PDX) of human prostate cancer adenocarcinoma. FIG. 2B is an immunoblot showing CDCP1 expression in in whole cell lysates of PDX of human SCNC. FIG. 2C is a plot showing the relative levels of total (full length and cleaved) cell surface CDCP1 in six human prostate cancer cell lines as determined by flow cytometry. Columns from androgen receptor (AR)- and PSMA-negative cell lines are shaded in gray. FIG. 2D shows the Bₘₐₓ (95% confidence intervals) values and R² values of saturation binding assays with ¹²⁵I-4A06 performed on PC3, DU145, and C4-2B cells.

FIG. 3A-3D show CDCP1 expression in vivo using ⁸⁹Zr-4A06 PET on prostate cancer tumors, including those with low or negligible PSMA expression. FIG. 3A is a line graph showing the radiotracer uptake in tumor and blood pool over time in intact male nu/nu mice bearing subcutaneous C4-2B tumors. The data were acquired using region of interest analysis of the PET data from a cohort of four mice imaged serially with PET/CT (*P<0.01). FIG. 3B are transaxial CT and PET/CT images showing the tumoral uptake of ⁸⁹Zr-4A06 at 48 hours post injection in two PSMA low adenocarcinoma and two SCNC tumor types. The arrows indicate the position of the subcutaneous tumor. FIG. 3C shows tumoral uptake of ⁸⁹Zr-4A06 at 48 hours post injection. The data were acquired from cohort sizes of n=4/tumor. FIG. 3D shows tumoral uptake of ⁶⁸Ga-PSMA 11 and ⁸⁹Zr-4A06 in mice bearing LTL-484 tumors at (n=4). Each mouse was imaged first with ⁶⁸Ga-PSMA 11, and then after 6 days, ⁸⁹Zr-4A06. The images were acquired 45 minutes post injection of ⁶⁸Ga-PSMA 11-, and 48-hours post injection of ⁸⁹Zr-4A06 (*P<0.01).

FIG. 4A-4C show that ¹⁷⁷Lu-4A06 monotherapy inhibits the growth of prostate cancer tumors. FIG. 4A shows the suppression of DU145 tumor growth by ¹⁷⁷Lu-4A06, administered in two fractions on day 0 and day 5, 400 μCi/dose. The volume of each tumor was normalized to the initial volume at day 0 of the study. The difference in tumor growth between treatment arms was significant, P<0.01. FIG. 4B shows the suppression of C4-2B tumor growth by ¹⁷⁷Lu-4A06, administered in two fractions on day 0 and day 5, 300 μCi/dose. The volume of each tumor was normalized to the initial volume at day 0 of the study. The difference in tumor growth between treatment arms was significant, P<0.01. FIG. 4C is a waterfall plot showing percent change in tumor volume for individual tumors from the C4-2B cohort. White bars represent tumors from mice receiving vehicle, while gray bars represent tumors from mice that received ¹⁷⁷Lu-4A06. For those bars that extend off the scale, the percent change in tumor volume is listed within the respective bar.

FIG. 5 shows the biodistribution of ⁸⁹Zr-4A06 at 48 hours post injection.

FIG. 6A-6B show relative ⁸⁹Zr-4A06 uptake in NSG mice bearing DU145, 22Rv1, LTL-545, LTL-331, or LTL-484 tumor xenografts. FIG. 6A shows the tumor to muscle ratios of ⁸⁹Zr-4A06 uptake. FIG. 6B shows the tumor to blood ratios of ⁸⁹Zr-4A06 uptake.

FIG. 7 shows percent change in body weight over time in animals treated with either ¹⁷⁷Lu-4A06 or vehicle.

FIG. 8 shows changes in tumor volume over time in each animal in the study, treated with either ¹⁷⁷Lu-4A06 or vehicle.

FIG. 9 is a spider plot showing changes in tumor volume over time in each mouse bearing a subcutaneous LTL-545 xenograft after treatment with either²²⁵Ac-4A06 or vehicle.

FIG. 10 shows fold changes in tumor volume over time in groups of mice bearing subcutaneous LTL-545 xenografts after treatment with either²²⁵Ac-4A06 or vehicle. Data are presented as mean±standard deviation.

FIG. 11 shows percent changes in body weight of each mouse bearing a subcutaneous LTL-545 xenograft after treatment with either²²⁵Ac-4A06 or vehicle. No mice were euthanized due to unsafe weight loss.

### DETAILED DESCRIPTION

The cell surface protein CUB domain containing protein 1 (CDCP1) has been found to be overexpressed in human prostate cancer cell lines and in biopsy tissues. However, studies described in the present disclosure now demonstrated that CDCP1 is a suitable target for metastatic castration-resistant prostate cancer (mCRPC), in both adenocarcinoma and neuroendocrine subtypes. Combined with low expression in normal human tissues, these data provide a compelling scientific rationale for translating CDCP1-directed radioligand therapy (RLT) into the clinic, either alone or in combination with other therapies, for treating advanced prostate cancer patients.

As described in Example 1, CDCP1 levels were evaluated using RNA-seq from 119 mCRPC biopsies. CDCP1 levels were assessed in 17 post enzalutamide or abiraterone treated mCRPC biopsies, 12 patient derived xenografts (PDX), and prostate cancer cell lines. 4A06, a recombinant human antibody that targets the CDCP1 ectodomain, was labeled with Zr-89 or Lu-177 and tested in tumor bearing mice.

CDCP1 expression was observed in 90% of mCRPC biopsies, including small cell neuroendocrine (SCNC) and adenocarcinomas with low FOLH1 (PSMA) levels. Fifteen of 17 evaluable mCRPC biopsies (85%) demonstrated membranous CDCP1 expression, and 4 of 17 (23%) had higher CDCP1 H-scores compared to PSMA. CDCP1 was expressed in ten of twelve PDX samples. Bmax values of ˜22,000, ˜6,200, and ˜2,800 fmol/mg were calculated for PC3, DU145, and C4-2B human prostate cancer cells respectively. ⁸⁹Zr-4A06 PET detected six human prostate cancer xenografts, including PSMA low tumors. ¹⁷⁷Lu-4A06 significantly suppressed growth of DU145 and C4-2B xenografts. These data provide the first evidence supporting CDCP1-directed RLT to treat mCRPC.

The disclosure relates to radioimmunotherapy for prostate cancer using an antibody or antibody fragment that specifically binds CUB domain-containing protein 1 (CDCP1). The disclosure further related to use of a diagnostic radioisotope-labeled anti-CDCP1 antibody or CDCP1-binding fragment thereof for radionuclide imaging of CDCP1 expression in vivo in prostate cancer patients.

### Definitions

As used herein and in the appended claims, the singular forms “a,” “an,” and “the” include plural references unless indicated otherwise. For example, “an” excipient includes one or more excipients.

The phrase “comprising” as used herein is open-ended, indicating that such embodiments may include additional elements. In contrast, the phrase “consisting of” is closed, indicating that such embodiments do not include additional elements (except for trace impurities). The phrase “consisting essentially of” is partially closed, indicating that such embodiments may further comprise elements that do not materially change the basic characteristics of such embodiments. It is understood that aspects and embodiments described herein as “comprising” include “consisting of” and “consisting essentially of” embodiments.

The term “about” as used herein in reference to a value, encompasses from 90% to 110% of that value (e.g., about 200 mCi radiolabeled antibody refers to 180 mCi to 220 mCi radiolabeled antibody and includes 200 mCi radiolabeled antibody).

The terms “individual” and “subject” refer to mammals. “Mammals” include, but are not limited to, humans, non-human primates (e.g., monkeys), farm animals, sport animals, rodents (e.g., mice and rats) and pets (e.g., dogs and cats). In some preferred embodiments, the subject is a human prostate cancer patient.

### 1. Anti-CDCP1 Antibodies

In one aspect, provided herein are antibodies and antibody fragments that specifically bind the CUB-domain containing protein 1 (CDCP1). The CDCP1 protein, which is also referred to in the literature as SIMA135, CD318 and TRASK (transmembrane and associated with src kinases), has a large extracellular domain of 665 amino acids and contains three CUB domains. The CUB domains mediate protein-protein interactions and are presumed to be involved in cell adhesion and interaction with the extracellular matrix (Bhatt et al., Oncogene, 24:5333-5343, 2005; and Casar et al., Oncogene, 33:255-268, 2014). For example, CDCP1 cellular adhesion plays a role in controlling phosphorylation status in at least keratinocytes. As described herein, CDCP1 expression appears to be regulated by RAS.

As used herein, CUB domain-containing protein 1 (CDCP1) is meant to include the extracellular portion of the full length CDCP1 protein. As is well known in the art, CDCP1 is a glycosylated transmembrane protein with an extracellular domain of approximately 638 amino acids. The full-length human version of CDCP1, after cleaving of the signal peptide (residues 1-29 of the full-length chain) is 807 amino acids in length. The amino acid sequence of human CDCP1 is found at Uniprot Record No. Q9H5V8, the entirely of which is incorporated by reference. As used herein, CDCP1 includes the human version and any natural variant thereof. For example, one natural variant of CDCP1 includes a Q525R mutation (with the numbering including the first 29 amino acids as the signal sequence that is subsequently cleaved), which is in the extracellular domain of the CDCP1. The antibody or antibody fragments of the present disclosure bind to the extracellular portion of the “mature” (lacking signal peptide) version of the CDCP1 protein.

As used herein, the term “antibody” refers to an immunoglobulin molecule that can specifically bind to a particular antigen. Antibodies have different isotypes or classes, such as but not limited to the isotypes known as IgA, IgD, IgE, IgG and IgM. The term antibody as used herein encompasses all isotypes. As is well understood in the art, a typical antibody is composed of two identical heavy chains and two identical light chains. The heavy chains are joined to one another via at least one disulfide bond and each light chain is joined to a heavy chain via a disulfide bond. Each heavy and light chain generally comprises a “variable domain” (VH and VL, respectively) at or near the N-terminus of the antibody. The variable domains for each chain are critical for antigen binding. The light chain contains one additional “constant region” (CL), and the heavy chain contains three or four additional constant regions (CH1, CH2, CH3, CH4). Thus, in specific embodiments, any of the antibodies provided herein may be IgA antibodies, IgD antibodies, IgE antibodies, IgG antibodies, or IgM antibodies. In other specific embodiments, any of the antibodies provided herein is a monoclonal antibody, e.g., comprising two identical light chains and two identical heavy chains.

The VH and VL chains generally each comprise three complementarity determining regions (CDRs) that determine antigen binding specificity. The CDRs can also be referred to as “loops” or “L regions.” The framework regions (FRs) are amino acid stretches within the VH and VL intervening between the CDRs. In full length VH and VL chains, each chain comprises three CDRs (Ls) and four framework regions (FR1 through FR4). The 4 FRs are separated respectively by the three CDRs (CDR1, CDR2, CDR3) or (L1, L2, L3). The CDRs, and in particular the CDR3 regions, and more particularly the VH CDR3, may, in certain embodiments, be largely responsible for antibody specificity.

A Fab fragment (fragment antigen-binding) is a region of an antibody that binds to antigens. Fab fragments may comprise one constant domain (CL and CH1) and one variable chain (VL and VH) of each of the heavy and the light chain. F(ab′)₂ refers to an antibody fragment comprising a Fab dimer. Fab and F(ab′)₂ may be generated by recombinant technology or by cleavage of an antibody or a fragment of antibody.

In some embodiments, the present disclosure provides monoclonal antibodies, or antigen-binding fragments thereof, which specifically bind to human CDCP1. The monoclonal antibody may be a human antibody, a humanized antibody, or a chimeric antibody, and may include a constant region. In some embodiments, the human constant region is selected from the group consisting of IgG1, IgG2, IgG3 and IgG4 constants regions. In some embodiments, the antigen-binding fragment is selected from the group consisting of Fab, Fab′-SH, F(ab′)₂, scFV and Fv fragments.

An antibody fragment may comprise part of an immunoglobulin molecule or a combination of parts of immunoglobulin molecules. In specific embodiments, the antibody fragments provided herein retain the ability to bind the same antigen that the full-length antibody binds, e.g., CDCP1, e.g., a CUB domain of CDCP1. The fragment may or may not bind to the exact same epitope as the full-length antibody from which the fragment is derived. Antibody fragments include but are not limited to F(ab′)₂, Fab, Fv and Fc fragment, as well as fusion peptide such as single chain Fv (scFv) fragments. ScFv fragments are single chain peptides that contain a VH chain and the VL chain, e.g., any of the VH and VL chains provided herein, linked to one another via a linker peptide. In specific embodiments, the connector peptide ranges from about two to about 50 amino acids. In some embodiments, the connector peptide ranges from about two to about 10 amino acids, from about 10 to about 15 amino acids, from about 15 to about 20 amino acids, from about 20 to about 25 amino acids, from about 25 to about 30 amino acids, from about 30 to about 35 amino acids, from about 35 to about 40 amino acids, from about 40 to about 45 amino acids or from about 45 to about 50 amino acids. The ScFv may retain the antigen binding ability of the original immunoglobulin molecule.

In select embodiments, the antibodies or antibody fragments provided herein comprise the variable heavy chain (VH) amino acid sequence of the Fab heavy chain of SEQ ID NO:1:

EISEVQLVES GGGLVQPGGS LRLSCAASGF NFSSSSIHWV RQAPGKGLEW VASISSSYGY
TYYADSVKGR FTISADTSKN TAYLQMNSLR AEDTAVYYCA RTVRGSKKPY FSGWAMDYWG
QGTLVTVSSA STKGPSVFPL APSSKSTSGG TAALGCLVKD YFPEPVTVSW NSGALTSGVH
TFPAVLQSSG LYSLSSVVTV PSSSLGTQTY ICNVNHKPSN TKVDKKVEPK SCDKTHTGGS
HHHHHH.

In other embodiments, the antibodies or antibody fragments of the present disclosure comprise the variable light chain (VL) amino acid sequence of the Fab heavy chain of SEQ ID NO:9:

SDIQMTQSPS SLSASVGDRV TITCRASQSV SSAVAWYQQK PGKAPKLLIY SASSLYSGVP
SRESGSRSGT DETLTISSLQ PEDFATYYCQ QSSYSLITFG QGTKVEIKRT VAAPSVFIFP
PSDSQLKSGT ASVVCLLNNF YPREAKVQWK VDNALQSGNS QESVTEQDSK DSTYSLSSTL
TLSKADYEKH KVYACEVTHQ GLSSPVTKSF NRGECGGSDY KDDDDK.

In still other embodiments, the antibodies or antibody fragments comprise both the VH of SEQ ID NO:1 and the VL of SEQ ID NO:9. The antibody or antibody fragment (i.e., CDCP1-binding fragment) containing the VH and VL of SEQ ID NO:1 and SEQ ID NO:9, respectively, is referred to herein as CDCP1-001. As used herein, the term “antibody fragment” refers to an “antigen-binding fragment” (i.e., CDCP1-binding fragment) of the designated antibody.

The underlined portions of the VH and VL amino acid sequences of the Fab heavy and light chains above represent the complementarity determining regions (CDRs) in each chain, as per the IMGT system (Lefranc et al., Dev Comp Immunol, 27:55-77, 2003). For example, CDR1 of the VH is the amino acid sequence FSSSSI (SEQ ID NO: 3), CDR2 of the VH is the amino acid sequence SISSSYGYTY (SEQ ID NO: 5) and CDR3 of the VH is the amino acid sequence is TVRGSKKPYFSGWAM (SEQ ID NO: 7). The non-underlined portions of the sequences above represent the framework regions (FRs). For example, FR1 of the VH is the amino acid sequence EISEVQLVESGGGLVQPGGSLRLSCAASGFN (SEQ ID NO:2), FR2 of the VH is the amino acid sequence HWVRQAPGKGLEWVA (SEQ ID NO:4), FR3 of the VH is the amino acid sequence YADSVKGRFTISADTSKNTAYLQMNSLRAEDTAVYYCAR (SEQ ID NO:6) and FR4 of the VH is the amino acid sequence DYWGQGTLVTVSS (SEQ ID NO:8). Likewise, CDR1 of VL is the amino acid sequence RASQSVSSAVA (SEQ ID NO:11), CDR2 of VL is the amino acid sequence SASSLYS (SEQ ID NO:13) and CDR3 of the VL is the amino acid sequence SSYSLI (SEQ ID NO:15). The FR1 of the VL is the amino acid sequence SDIQMTQSPSSLSASVGDRVTITC (SEQ ID NO:10), FR2 of VL is the amino acid sequence WYQQKPGKAPKLLIY (SEQ ID NO:12), FR3 of VL is the amino acid sequence GVPSRFSGSRSGTDFTLTISSLQPEDFATYYCQQ (SEQ ID NO:14) and FR4 of the VL is the amino acid sequence TFGQGTKVEIK (SEQ ID NO:16). In the exemplary Fab heavy and light chain sequences shown above, DKTHTGGSHHHHHH (SEQ ID NO:79) and GGSDYKDDDDK (SEQ ID NO:80) are affinity tags.

In other embodiments, the antibodies or antibody fragments comprise VH FR1-FR4, VL FR1-FR4 and VL CDR1-CDR2 of the CDCP1-001 fragment disclosed above, and different VH CDR1-CDR3 and VL CDR3, as shown below in Table A-1. In another embodiment, the antibodies or antibody fragments comprise VL CDR1-CDR2 of the CDCP1-001 fragment, and different VH CDR1-CDR3 VL CDR3, as shown below in Table A-1. In other words, in these specific embodiments, the antibodies or antibody fragments CDCP1-002 through CDCP1-011 would possess the VH CDRs and the VL CDR3 listed in Table A-1, respectively, plus the VL CDR1 (SEQ ID NO:11) and VL CDR2 (SEQ ID NO:13) from the CDCP1-001 fragment. For example, in one embodiment, the VH CDCP1-002 comprises the amino acid sequence from N-terminus to C-terminus: SEQ ID NO:2 (VH FR1), SEQ ID NO: 17 (VH CDR1), SEQ ID NO:4 (VH FR2), SEQ ID NO:18 (VH CDR2), SEQ ID NO:6 (VH FR3), SEQ ID NO:19 (VH CDR3) and SEQ ID NO:8 (VH FR4). Similarly, in one embodiment, the VL CDCP1-002 comprises the amino acid sequence from N-terminus to C-terminus: SEQ ID NO:10 (VL FR1), SEQ ID NO: 11 (VH CDR1), SEQ ID NO:12 (VH FR2), SEQ ID NO:13 (VH CDR2), SEQ ID NO:14 (VH FR3), SEQ ID NO:20 (VH CDR3) and SEQ ID NO:16 (VH FR4). The combination of VH FRs1-4 and VH CDRs1-3, in proper order, make up the VH chain for each of the 001-011 anti-CDCP1 antibody fragments. Similarly, the combination of VL FRs1-4 and VL CDRs1-3, in proper order, make up the VL chain for each of the 001-011 anti-CDCP1 antibody fragments.

TABLE A-1
Sequences of CDRs of Anti-CDCP1 Fabs

| Fab Region | CDR1 | CDR2 | CDR3 |
|---|---|---|---|
| CDCP1-001 V_{H} | FSSSSI | SISSSYGYTY | TVRGSKKPYFSGWAM |
| | (SEQ ID NO: 3) | (SEQ ID NO: 5) | (SEQ ID NO: 7) |
| CDCP1-001 V_{L} | RASQSVSSAVA | SASSLYS | SSYSLI |
| | (SEQ ID NO: 11) | (SEQ ID NO: 13) | (SEQ ID NO: 15) |
| CDCP1-002 V_{H} | LSYYYI | SIYSSSSYTS | AYYGF |
| | (SEQ ID NO: 17) | (SEQ ID NO: 18) | (SEQ ID NO: 19) |
| CDCP1-002 V_{L} | RASQSVSSAVA | SASSLYS | SYYYYPI |
| | (SEQ ID NO: 11) | (SEQ ID NO: 13) | (SEQ ID NO: 20) |
| CDCP1-003 V_{H} | ISYYSM | SISPYSGYTS | GYYAL |
| | (SEQ ID NO: 21) | (SEQ ID NO: 22) | (SEQ ID NO: 23) |
| CDCP1-003 V_{L} | RASQSVSSAVA | SASSLYS | YYYFYPF |
| | (SEQ ID NO: 11) | (SEQ ID NO: 13) | (SEQ ID NO: 24) |
| CDCP1-004 V_{H} | ISYYYM | SIYSSYGYTS | VYYGF |
| | (SEQ ID NO: 25) | (SEQ ID NO: 26) | (SEQ ID NO: 27) |
| CDCP1-004 V_{L} | RASQSVSSAVA | SASSLYS | SYYVYPI |
| | (SEQ ID NO: 11) | (SEQ ID NO: 13) | (SEQ ID NO: 28) |
| CDCP1-005 V_{H} | ISYYYI | SIYPYYGSTY | AYYGF |
| | (SEQ ID NO: 29) | (SEQ ID NO: 30) | (SEQ ID NO: 31) |
| CDCP1-005 V_{L} | RASQSVSSAVA | SASSLYS | SYWSFPI |
| | (SEQ ID NO: 11) | (SEQ ID NO: 13) | (SEQ ID NO: 32) |
| CDCP1-006 V_{H} | LYYSYM | YISPYSGSTY | YSYSAL |
| | (SEQ ID NO: 33) | (SEQ ID NO: 34) | (SEQ ID NO: 35) |
| CDCP1-006 V_{L} | RASQSVSSAVA | SASSLYS | SSWHYHLF |
| | (SEQ ID NO: 11) | (SEQ ID NO: 13) | (SEQ ID NO: 36) |
| CDCP1-007 V_{H} | IYSYYI | SIYPYYGYTS | AYYGM |
| | (SEQ ID NO: 37) | (SEQ ID NO: 38) | (SEQ ID NO: 39) |
| CDCP1-007 V_{L} | RASQSVSSAVA | SASSLYS | SYFYWPI |
| | (SEQ ID NO: 11) | (SEQ ID NO: 13) | (SEQ ID NO: 40) |
| CDCP1-008 V_{H} | ISSYYM | SIYPYSGYTY | AYYAM |
| | (SEQ ID NO: 41) | (SEQ ID NO: 42) | (SEQ ID NO: 43) |
| CDCP1-008 V_{L} | RASQSVSSAVA | SASSLYS | SYYVYPI |
| | (SEQ ID NO: 11) | (SEQ ID NO: 13) | (SEQ ID NO: 44) |
| CDCP1-009 V_{H} | LYSYYI | SIYPYYSSTS | YYYAM |
| | (SEQ ID NO: 45) | (SEQ ID NO: 46) | (SEQ ID NO: 47) |
| CDCP1-009 V_{L} | RASQSVSSAVA | SASSLYS | GYAGSWHPI |
| | (SEQ ID NO: 11) | (SEQ ID NO: 13) | (SEQ ID NO: 48) |
| CDCP1-010 V_{H} | IYSYSM | SISPYYSYTS | AYYAL |
| | (SEQ ID NO: 49) | (SEQ ID NO: 50) | (SEQ ID NO: 51) |
| CDCP1-010 V_{L} | RASQSVSSAVA | SASSLYS | SYWYYPI |
| | (SEQ ID NO: 11) | (SEQ ID NO: 13) | (SEQ ID NO: 52) |
| CDCP1-011 V_{H} | ISYYYM | SIYSSSSYTS | SYYAM |
| | (SEQ ID NO: 53) | (SEQ ID NO: 54) | (SEQ ID NO: 55) |
| CDCP1-011 V_{L} | RASQSVSSAVA | SASSLYS | SYYVYPI |
| | (SEQ ID NO: 11) | (SEQ ID NO: 13) | (SEQ ID NO: 56) |

TABLE A-2
Sequences of Variable Regions of Anti-CDCP1 Fabs^{∧}
Fab Variable Region
CDCP1-002 V_{H} (SEQ ID NO: 57)
EVQLVES GGGLVQPGGS LRLSCAASGF NLSYYYIHWV RQAPGKGLEW VASIYSSSSY
TSYADSVKGR FTISADTSKN TAYLQMNSLR AEDTAVYYCA RAYYGFDYWG QGTLVTVSS
CDCP1-002 V_{L} (SEQ ID NO: 58)
DIQMTQSPS SLSASVGDRV TITCRASQSV SSAVAWYQQK PGKAPKLLIY SASSLYSGVP
SRFSGSRSGT DFTLTISSLQ PEDFATYYCQ QSYYYYPITFG QGTKVEIK
CDCP1-003 V_{H} (SEQ ID NO: 59)
EVQLVES GGGLVQPGGS LRLSCAASGF NISYYSMHWV RQAPGKGLEW VASISPYSGY
TSYADSVKGR FTISADTSKN TAYLQMNSLR AEDTAVYYCA RGYYALDYWG QGTLVTVSS
CDCP1-003 V_{L} (SEQ ID NO: 60)
DIQMTQSPS SLSASVGDRV TITCRASQSV SSAVAWYQQK PGKAPKLLIY SASSLYSGVP
SRFSGSRSGT DFTLTISSLQ PEDFATYYCQ QYYYFYPFTFG QGTKVEIK
CDCP1-004 V_{H} (SEQ ID NO: 61)
EVQLVES GGGLVQPGGS LRLSCAASGF NISYYYMHWV RQAPGKGLEW VASIYSSYGY
TSYADSVKGR FTISADTSKN TAYLQMNSLR AEDTAVYYCA RVYYGFDYWG QGTLVTVSS
CDCP1-004 V_{L} (SEQ ID NO: 62)
DIQMTQSPS SLSASVGDRV TITCRASQSV SSAVAWYQQK PGKAPKLLIY SASSLYSGVP
SRFSGSRSGT DFTLTISSLQ PEDFATYYCQ QSYYVYPITFG QGTKVEIK
CDCP1-005 V_{H} (SEQ ID NO: 63)
EVQLVES GGGLVQPGGS LRLSCAASGF NISYYYIHWV RQAPGKGLEW VASIYPYYGS
TYYADSVKGR FTISADTSKN TAYLQMNSLR AEDTAVYYCA RAYYGFDYWG QGTLVTVSS
CDCP1-005 V_{L} (SEQ ID NO: 64)
DIQMTQSPS SLSASVGDRV TITCRASQSV SSAVAWYQQK PGKAPKLLIY SASSLYSGVP
SRFSGSRSGT DFTLTISSLQ PEDFATYYCQ QSYWSFPITFG QGTKVEIK
CDCP1-006 V_{H} (SEQ ID NO: 65)
EVQLVES GGGLVQPGGS LRLSCAASGF NLYYSYMHWV RQAPGKGLEW VAYISPYSGS
TYYADSVKGR FTISADTSKN TAYLQMNSLR AEDTAVYYCA RYSYSALDYWG QGTLVTVSS
CDCP1-006 V_{L} (SEQ ID NO: 66)
DIQMTQSPS SLSASVGDRV TITCRASQSV SSAVAWYQQK PGKAPKLLIY SASSLYSGVP
SRFSGSRSGT DFTLTISSLQ PEDFATYYCQ QSSWHYHLFTFG QGTKVEIK
CDCP1-007 V_{H} (SEQ ID NO: 67)
EVQLVES GGGLVQPGGS LRLSCAASGF NIYSYYIHWV RQAPGKGLEW VASIYPYYGY
TSYADSVKGR FTISADTSKN TAYLQMNSLR AEDTAVYYCA RAYYGMDYWG QGTLVTVSS
Fab Variable Region
CDCP1-007 V_{L} (SEQ ID NO: 68)
DIQMTQSPS SLSASVGDRV TITCRASQSV SSAVAWYQQK PGKAPKLLIY SASSLYSGVP
SRFSGSRSGT DFTLTISSLQ PEDFATYYCQ QSYFYWPITFG QGTKVEIK
CDCP1-008 V_{H} (SEQ ID NO: 69)
EVQLVES GGGLVQPGGS LRLSCAASGF NISSYYMHWV RQAPGKGLEW VASIYPYSGY
TYYADSVKGR FTISADTSKN TAYLQMNSLR AEDTAVYYCA RAYYAMDYWG QGTLVTVSS
CDCP1-008 V_{L} (SEQ ID NO: 70)
DIQMTQSPS SLSASVGDRV TITCRASQSV SSAVAWYQQK PGKAPKLLIY SASSLYSGVP
SRFSGSRSGT DFTLTISSLQ PEDFATYYCQ QSYYVYPITFG QGTKVEIK
CDCP1-009 V_{H} (SEQ ID NO: 71)
EVQLVES GGGLVQPGGS LRLSCAASGF NLYSYYIHWV RQAPGKGLEW VASIYPYYSS
TSYADSVKGR FTISADTSKN TAYLQMNSLR AEDTAVYYCA RYYYAMDYWG QGTLVTVSS
CDCP1-009 V_{L} (SEQ ID NO: 72)
DIQMTQSPS SLSASVGDRV TITCRASQSV SSAVAWYQQK PGKAPKLLIY SASSLYSGVP
SRFSGSRSGT DFTLTISSLQ PEDFATYYCQ QGYAGSWHPITFG QGTKVEIK
CDCP1-010 V_{H} (SEQ ID NO: 73)
EVQLVES GGGLVQPGGS LRLSCAASGF NIYSYSMHWV RQAPGKGLEW VASISPYYSY
TSYADSVKGR FTISADTSKN TAYLQMNSLR AEDTAVYYCA RAYYALDYWG QGTLVTVSS
CDCP1-010 V_{L} (SEQ ID NO: 74)
DIQMTQSPS SLSASVGDRV TITCRASQSV SSAVAWYQQK PGKAPKLLIY SASSLYSGVP
SRFSGSRSGT DFTLTISSLQ PEDFATYYCQ QSYWYYPITFG QGTKVEIK
CDCP1-011 V_{H} (SEQ ID NO: 75)
EVQLVES GGGLVQPGGS LRLSCAASGF NISYYYMHWV RQAPGKGLEW VASIYSSSSY
TSYADSVKGR FTISADTSKN TAYLQMNSLR AEDTAVYYCA RSYYAMDYWG QGTLVTVSS
CDCP1-011 V_{L} (SEQ ID NO: 76)
DIQMTQSPS SLSASVGDRV TITCRASQSV SSAVAWYQQK PGKAPKLLIY SASSLYSGVP
SRFSGSRSGT DFTLTISSLQ PEDFATYYCQ QSYYVYPITFG QGTKVEIK
CDCP1-001 V_{H} (SEQ ID NO: 77)
EVQLVES GGGLVQPGGS LRLSCAASGF NFSSSSIHWV RQAPGKGLEW VASISSSYGY
TYYADSVKGR FTISADTSKN TAYLQMNSLR AEDTAVYYCA RTVRGSKKPY FSGWAMDYWG
QGTLVTVSS
CDCP1-001 V_{L} (SEQ ID NO: 78)
DIQMTQSPS SLSASVGDRV TITCRASQSV SSAVAWYQQK PGKAPKLLIY SASSLYSGVP
SRFSGSRSGT DFTLTISSLQ PEDFATYYCQ QSSYSLITFG QGTKVEIK
^{∧}CDR sequences are underlined

Similarly, the combination of any 4 VH FR regions with VH CDRs1-3 from Table A-1, in proper order, would make up a VH chain for an anti-CDCP1 antibody or antibody fragment that is within the scope of the present disclosure. Furthermore, the combination of any 4 VL FRs1, VL CDR1 (SEQ ID NO:11), CDR2 (SEQ ID NO:13) and VL CDR3 from Table A-1, in proper order, make up the VL chain for an anti-CDCP1 antibody or antibody fragment that is within the scope of the present disclosure. Accordingly, the disclosure provides humanized antibodies or antibody fragments. For example, humanized antibodies or antibody fragments may be generated by inserting CDRs generated in animals into framework regions from other human antibodies or antibody fragments. Thus, the framework regions of the antibody or antibody fragments need not be the identical amino acid sequences of the framework regions of the CDCP1-001 antibody.

In certain embodiments, an antibody or antibody fragment provided herein comprises:
the VH amino acid sequence shown in SEQ ID NO:57, and the VL amino acid sequence shown in SEQ ID NO:58;
the VH amino acid sequence shown in SEQ ID NO:59, and the VL amino acid sequence shown in SEQ ID NO:60;
the VH amino acid sequence shown in SEQ ID NO:61, and the VL amino acid sequence shown in SEQ ID NO:62;
the VH amino acid sequence shown in SEQ ID NO:63, and the VL amino acid sequence shown in SEQ ID NO:64;
the VH amino acid sequence shown in SEQ ID NO:65, and the VL amino acid sequence shown in SEQ ID NO:66;
the VH amino acid sequence shown in SEQ ID NO:67, and the VL amino acid sequence shown in SEQ ID NO:68;
the VH amino acid sequence shown in SEQ ID NO:69, and the VL amino acid sequence shown in SEQ ID NO:70;
the VH amino acid sequence shown in SEQ ID NO:71, and the VL amino acid sequence shown in SEQ ID NO:72;
the VH amino acid sequence shown in SEQ ID NO:73, and the VL amino acid sequence shown in SEQ ID NO:74;
the VH amino acid sequence shown in SEQ ID NO:75, and the VL amino acid sequence shown in SEQ ID NO:76; or
the VH amino acid sequence shown in SEQ ID NO:77, and the VL amino acid sequence shown in SEQ ID NO:78.

In certain embodiments, an antibody or antibody fragment provided herein comprises the VH CDR1, CDR2, and CDR3 amino acid sequences shown in SEQ ID NO:1 above, combined with the VL CDR1, CDR2 and CDR3 sequences shown in SEQ ID NO:9 above. Optionally, the VL CDR3 shown in SEQ ID NO:9 may be substituted with one of the VL CDR3 sequences shown in Table A-1. Thus, in specific embodiments, provided herein are antibodies or antibody fragments that comprise:
VH CDR1, CDR2 and CDR3 comprising the amino acid sequence of SEQ ID NO:3, SEQ ID NO:5 and SEQ ID NO:7, and VL CDR1, CDR2 and CDR3 comprising the amino acid sequence of SEQ ID NO:11, SEQ ID NO:13 and SEQ ID NO:15;
VH CDR1, CDR2 and CDR3 comprising the amino acid sequence of SEQ ID NO:17, SEQ ID NO:18 and SEQ ID NO:19, and VL CDR1, CDR2 and CDR3 comprising the amino acid sequence of SEQ ID NO:11, SEQ ID NO:13 and SEQ ID NO:20;
VH CDR1, CDR2 and CDR3 comprising the amino acid sequence of SEQ ID NO:21, SEQ ID NO:22 and SEQ ID NO:23, and VL CDR1, CDR2 and CDR3 comprising the amino acid sequence of SEQ ID NO:11, SEQ ID NO:13 and SEQ ID NO:24;
VH CDR1, CDR2 and CDR3 comprising the amino acid sequence of SEQ ID NO:25, SEQ ID NO:26 and SEQ ID NO:27, and VL CDR1, CDR2 and CDR3 comprising the amino acid sequence of SEQ ID NO:11, SEQ ID NO:13 and SEQ ID NO:28;
VH CDR1, CDR2 and CDR3 comprising the amino acid sequence of SEQ ID NO 29, SEQ ID NO:30 and SEQ ID NO:31, and VL CDR1, CDR2 and CDR3 comprising the amino acid sequence of SEQ ID NO:11, SEQ ID NO:13 and SEQ ID NO:32;
VH CDR1, CDR2 and CDR3 comprising the amino acid sequence of SEQ ID NO:33, SEQ ID NO:34 and SEQ ID NO:35, and VL CDR1, CDR2 and CDR3 comprising the amino acid sequence of SEQ ID NO:11, SEQ ID NO:13 and SEQ ID NO:36;
VH CDR1, CDR2 and CDR3 comprising the amino acid sequence of SEQ ID NO:37, SEQ ID NO:38 and SEQ ID NO:39, and VL CDR1, CDR2 and CDR3 comprising the amino acid sequence of SEQ ID NO:11, SEQ ID NO:13 and SEQ ID NO:40;
VH CDR1, CDR2 and CDR3 comprising the amino acid sequence of SEQ ID NO:41, SEQ ID NO:42 and SEQ ID NO:43, and VL CDR1, CDR2 and CDR3 comprising the amino acid sequence of SEQ ID NO:11, SEQ ID NO:13 and SEQ ID NO:44;
VH CDR1, CDR2 and CDR3 comprising the amino acid sequence of SEQ ID NO:45, SEQ ID NO:46 and SEQ ID NO:47, and VL CDR1, CDR2 and CDR3 comprising the amino acid sequence of SEQ ID NO:11, SEQ ID NO:13 and SEQ ID NO:48;
VH CDR1, CDR2 and CDR3 comprising the amino acid sequence of SEQ ID NO:49, SEQ ID NO:50 and SEQ ID NO:51, and VL CDR1, CDR2 and CDR3 comprising the amino acid sequence of SEQ ID NO:11, SEQ ID NO:13 and SEQ ID NO:52; or
VH CDR1, CDR2 and CDR3 comprising the amino acid sequence of SEQ ID NO:53, SEQ ID NO:54 and SEQ ID NO:55, and VL CDR1, CDR2 and CDR3 comprising the amino acid sequence of SEQ ID NO:11, SEQ ID NO:13 and SEQ ID NO:56.

In some embodiments, an antibody or antibody fragment provided herein comprises:
the VH amino acid sequence selected from the group consisting of SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, and SEQ ID NO:77; and
the VL amino acid sequence selected from the group consisting of SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, and SEQ ID NO:78. For instance, an antibody or antibody fragment provided herein may comprise the VH amino acid sequence of SEQ ID NO:57, and the VL amino acid sequence selected from the group consisting of SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, and SEQ ID NO:78. Similarly, an antibody or antibody fragment provided herein may comprise the VL amino acid sequence of SEQ ID NO:58, and the VH amino acid sequence selected from the group consisting of SEQ ID NO:1, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, and SEQ ID NO:77.

In other embodiments, any of the antibodies provided herein may be a part of a bispecific or multispecific antibody. The bispecific or multispecific antibody may have both, or all, binding domains specific for CDCP1 (e.g., a CUB domain of CDCP1). For instance, a bispecific antibody may comprise VH and VL from any combination of two of the anti-CDCP1-specific Fab sequences shown in Table A-2. Alternatively, the bispecific or multispecific antibody may have a single binding domain specific for CDCP1 (e.g., a CUB domain of CDCP1), and one or more domains specific for a second antigen. In similar fashion, a bispecific antibody can comprise VH and VL from any other combination two of the anti-CDCP1-specific Fab sequences provided herein. The CDCP1-binding domains of the present disclosure may be contained in other antibody formats, including but not limited to monospecific Fab2, bispecific Fab2, trispecific Fab3, monovalent IgGT, bispecific diabody, trispecific triabody, scFv-Fc, minibody, etc.

In still further embodiments, the CDCP1-binding domains of the present disclosure may form part of a “T-cell engager” comprising a CD3 binding domain for a linking a T cell to a CDCP1+ cancer cell (anti-CDCP1×anti-CD3). Alternatively, the CDCP1-binding domains of the present disclosure may form part of a “NK-cell engager” comprising a NKG2D or CD16 binding domain for linking a NK cell to a CDCP1+ cancer cell (anti-CDCP1×anti-NKG2D or anti-CD16). Additionally, the CDCP1-binding domains of the present disclosure may form part of an ectodomain of a chimeric T cell receptor.

As used herein with respect to polypeptides, the term “substantially pure” means that the polypeptides are essentially free of other substances with which they may be found in nature or in vivo systems to an extent practical and appropriate for their intended use. In particular, the polypeptides are sufficiently pure and are sufficiently free from other biological constituents of their host cells so as to be useful in, for example, generating antibodies, sequencing, or producing pharmaceutical preparations. By techniques well known in the art, substantially pure polypeptides may be produced in light of the polynucleotide and amino acid sequences disclosed herein. Because a substantially purified polypeptide of the disclosure may be admixed with a pharmaceutically acceptable carrier in a pharmaceutical preparation, the polypeptide may comprise only a certain percentage by weight of the preparation. The polypeptide is nonetheless substantially pure in that it has been substantially separated from the substances with which it may be associated in living systems.

As used herein, “sequence identity” is a measure of the identity of nucleotide sequences or amino acid sequences compared to a reference nucleotide or amino acid sequence. A polypeptide having an amino acid sequence at least, for example, about 95% “sequence identity” to a reference an amino acid sequence, e.g., SEQ ID NO: 1, is understood to mean that the amino acid sequence of the polypeptide is identical to the reference sequence except that the amino acid sequence may include up to about five modifications per each 100 amino acids of the reference amino acid sequence. In other words, to obtain a peptide having at least about 95% sequence identity to a reference amino acid sequence, up to about 5% of the amino acid residues of the reference sequence may be deleted or substituted with another amino acid or a number of amino acids up to about 5% of the total amino acids in the reference sequence may be inserted into the reference sequence. These modifications of the reference sequence may occur at the N-terminus or C-terminus positions of the reference amino acid sequence or anywhere between those terminal positions, interspersed either individually among amino acids in the reference sequence or in one or more contiguous groups within the reference sequence.

In general, the sequences are aligned so that the highest order match is obtained. “Sequence identity” per se has an art-recognized meaning and can be calculated using well known techniques. In one embodiment of the present disclosure, the algorithm used to determine sequence identity between two or more polypeptides is BLASTP. In another embodiment of the present disclosure, the algorithm used to determine sequence identity between two or more polypeptides is FASTDB (Brutlag, Comp. App. Biosci. 6:237-245, 1990). In a FASTDB sequence alignment, the query and reference sequences are amino sequences. The result of sequence alignment is in percent sequence identity. In one embodiment, parameters that may be used in a FASTDB alignment of amino acid sequences to calculate percent sequence identity include, but are not limited to: Matrix=PAM, k-tuple=2, Mismatch Penalty=1, Joining Penalty=20, Randomization Group Length=0, Cutoff Score=1, Gap Penalty=5, Gap Size Penalty 0.05, Window Size=500 or the length of the subject amino sequence, whichever is shorter.

If the reference sequence is shorter or longer than the query sequence because of N-terminus or C-terminus additions or deletions, but not because of internal additions or deletions, a manual correction can be made, because the FASTDB program does not account for N-terminus and C-terminus truncations or additions of the reference sequence when calculating percent sequence identity. For query sequences truncated at the N- or C-termini, relative to the reference sequence, the percent sequence identity is corrected by calculating the number of residues of the query sequence that are N- and C-terminus to the reference sequence that are not matched/aligned, as a percent of the total bases of the query sequence. The results of the FASTDB sequence alignment determine matching/alignment. The alignment percentage is then subtracted from the percent sequence identity, calculated by the above FASTDB program using the specified parameters, to arrive at a final percent sequence identity score. This corrected score can be used for the purposes of determining how alignments “correspond” to each other, as well as percentage sequence identity. Residues of the reference sequence that extend past the N- or C-termini of the query sequence may be considered for the purposes of manually adjusting the percent sequence identity score. That is, residues that are not matched/aligned with the N- or C-termini of the comparison sequence may be counted when manually adjusting the percent sequence identity score or alignment numbering.

For example, a 90 amino acid residue query sequence is aligned with a 100 residue reference sequence to determine percent identity. The deletion occurs at the N-terminus of the query sequence and therefore, the FASTDB alignment does not show a match/alignment of the first 10 residues at the N-terminus. The 10 unpaired residues represent 10% of the reference sequence (number of residues at the N- and C-termini not matched/total number of residues in the reference sequence) so 10% is subtracted from the percent sequence identity score calculated by the FASTDB program. If the remaining 90 residues were perfectly matched (100% alignment) the final percent sequence identity would be 90% (100% alignment−10% unmatched overhang). In another example, a 90 residue query sequence is compared with a 100 reference sequence, except that the deletions are internal deletions. In this case the percent sequence identity calculated by FASTDB is not manually corrected, since there are no residues at the N- or C-termini of the subject sequence that are not matched/aligned with the query. In still another example, a 110 amino acid query sequence is aligned with a 100 residue reference sequence to determine percent sequence identity. The addition in the query occurs at the N-terminus of the query sequence and therefore, the FASTDB alignment may not show a match/alignment of the first 10 residues at the N-terminus. If the remaining 100 amino acid residues of the query sequence have 95% sequence identity to the entire length of the reference sequence, the N-terminal addition of the query would be ignored and the percent identity of the query to the reference sequence would be 95%.

As used here, the term “conservative substitution” denotes the replacement of an amino acid residue by another biologically similar residue. Conservative substitution for this purpose may be defined as set out in the tables below. Amino acids can be classified according to physical properties and contribution to secondary and tertiary protein structure as shown in Table B and Table C. A conservative substitution is recognized in the art as a substitution of one amino acid for another amino acid that has similar properties, such as a substitution of one amino acid for another amino acid in the same row of Table B or Table C. Exemplary conservative substitutions are set out below in Table D.

TABLE B
Amino Acid Classification-5 Groups

| Side Chain Characteristic | Amino Acid |
|---|---|
| Aliphatic | |
| Non-polar | Gly, Ala, Pro, Iso, Leu, Val |
| Polar-uncharged | Cys, Ser, Thr, Met, Asn, Gln |
| Polar-charged | Asp, Glu, Lys, Arg |
| Aromatic | His, Phe, Trp, Tyr |
| Other | Asn, Gln, Asp, Glu |

TABLE C
Amino Acid Classification-10 Groups

| Side Chain Characteristic | Amino Acid |
|---|---|
| Non-polar (hydrophobic) | |
| Aliphatic: | Ala, Leu, Iso, Val, Pro |
| Aromatic: | Phe, Trp |
| Sulfur-containing: | Met |
| Borderline: | Gly |
| Uncharged-polar | |
| Hydroxyl: | Ser, Thr, Tyr |
| Amides: | Asn, Gln |
| Sulfhydryl: | Cys |
| Borderline: | Gly |
| Positively Charged (Basic): | Lys, Arg, His |
| Negatively Charged (Acidic) | Asp, Glu |

TABLE D
Conservative Amino Acid Substitutions

| Original Residue | Exemplary Substitution |
|---|---|
| Ala (A) | Val, Leu, Ile |
| Arg (R) | Lys, Gln, Asn |
| Asn (N) | Gln, His, Lys, Arg |
| Asp (D) | Glu |
| Cys (C) | Ser |
| Gln (Q) | Asn |
| Glu (E) | Asp |
| His (H) | Asn, Gln, Lys, Arg |
| Ile (I) | Leu, Val, Met, Ala, Phe |
| Leu (L) | Ile, Val, Met, Ala, Phe |
| Lys (K) | Arg, Gln, Asn |
| Met (M) | Leu, Phe, Ile |
| Phe (F) | Leu, Val, Ile, Ala |
| Pro (P) | Gly |
| Ser (S) | Thr |
| Thr (T) | Ser |
| Trp (W) | Tyr |
| Tyr (Y) | Trp, Phe, Thr, Ser |
| Val (V) | Ile, Leu, Met, Phe, Ala |

In select embodiments, the disclosure is directed to antibodies or antibody fragments where the amino acid sequence of one or more framework regions is mutated. In one specific embodiment, the mutations in the one or more framework regions is a conserved substitution. In more specific embodiments, the antibodies or antibody fragments of the present disclosure comprise an amino acid sequence having at least 80%, 85%, 90%, 95%, 96%, 97%, 98%, 99% or 100% sequence identity to any one of the amino acid sequences of SEQ ID NO:2, SEQ ID NO:4, SEQ ID NO:6, SEQ ID NO:8, SEQ ID NO: 10, SEQ ID NO: 12, SEQ ID NO: 14, and SEQ ID NO: 16.

Likewise, in some embodiments, the disclosure is directed to antibodies or antibody fragments that have a high level of identity to the VH and VL amino acid sequences of any one of exemplary antibodies CDCP1-001 to CDCP1-011. In specific embodiments, the antibodies or antibody fragments of the present disclosure comprise an amino acid sequence having at least 90%, 95%, 96%, 97%, 98%, or 99% sequence identity to any one of the amino acid sequences of SEQ ID NOs:57-78.

### 2. Radiolabeled Antibodies

The disclosure also provides for antibodies or antibody fragments that are labeled with a radionuclide (also referred to herein as a radioisotope). In some embodiments, the disclosure provides antibody-radionuclide conjugates (ARCs). As used herein, an ARC need not be composed of an entire, intact antibody and instead can be composed of any one of the antibody fragments disclosed herein. In some embodiments, the radioisotope-labeled antibody or antibody fragment has the formula: Radioisotope-Linker-Antibody (R*-L-Ab), wherein R* is a radioisotope, L is a linker, and Ab is an antibody or antibody fragment that specifically binds CUB domain-containing protein 1 (CDCP1) ectodomain. In some embodiments, the linker is a macrocyclic chelator. In an exemplary embodiment, the macrocyclic chelator comprises tetraxetan. In some embodiments, the radioisotope is a therapeutic radioisotope. In other embodiments, the radioisotope is a diagnostic radioisotope.

As used herein, the term “therapeutic radioisotope” refers to an alpha-emitter and/or a beta-minus-emitter, preferably with a half-life of hours to days. In some embodiments, the therapeutic radioisotope is selected from the group consisting of ⁶⁷Cu, ⁹⁰Y, ¹³¹I, ¹⁵³Sm, ¹⁷⁷Lu, ²¹¹At, ²¹²Pb, ²²³Ra, ²²⁵Ac, and ²²⁷Th. In an exemplary embodiment, the therapeutic radioisotope is ¹⁷⁷Lu.

The term “diagnostic radioisotope” as used herein, refers to a positron-emitter, preferably with a half-life of minutes to hours. In some embodiments, the diagnostic radioisotope is selected from the group consisting of ¹⁸F, ⁴⁴Sc ⁶⁴Cu, ⁶⁸Ga, ⁸⁹Zr, and ¹²⁴I. In an exemplary embodiment, the diagnostic radioisotope is ⁸⁹Zr.

In some preferred embodiments, the antibody or antibody fragment comprises the amino acid sequences of the light chain CDRs of 4A06 are set forth as SEQ ID NO:11, SEQ ID NO:13 and SEQ ID NO:20, and the amino acid sequences of the heavy chain CDRs of 4A06 are set forth as SEQ ID NO:17, SEQ ID NO:18, and SEQ ID NO:19. In some preferred embodiments, the antibody or antibody fragment comprises the amino acid sequence of the heavy chain variable domain (VH) of 4A06 are set forth as SEQ ID NO:57, and the amino acid sequence of the light chain variable domain (VL) of 4A06 is set forth as SEQ ID NO:58. The recombinant monoclonal antibody 4A06 is also referred to herein as CDCP1-002. In some embodiments, the antibody is an IgG₁ antibody.

### 3. Pharmaceutical Compositions

Further provided are pharmaceutical compositions comprising the radiolabeled antibodies or antibody fragments described. The pharmaceutical compositions comprise at least one radiolabeled antibody or antibody fragment of the present disclosure and a pharmaceutical excipient. The pharmaceutical compositions may be administered, or may be formulated to be administered, by parenteral administration, for example, intravenous administration. Intravenous administration may be done by injection or infusion.

The present compositions will contain a therapeutically effective amount of the radiolabeled antibodies or antibody fragments, together with a suitable amount of a pharmaceutically acceptable vehicle so as to provide the form for proper administration to a patient.

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “vehicle” refers to a diluent, adjuvant, excipient, or carrier with which a compound of the disclosure is administered. Such pharmaceutical vehicles can be liquids, such as water and oils, including those of petroleum, animal, vegetable or synthetic origin, such as peanut oil, soybean oil, mineral oil, sesame oil and the like. The pharmaceutical vehicles can be saline, gum acacia, gelatin, starch paste, talc, keratin, colloidal silica, urea, and the like. In addition, auxiliary, stabilizing, thickening, lubricating and coloring agents may be used. When administered to a patient, the compounds of the disclosure and pharmaceutically acceptable vehicles should be sterile. Water is one example of a vehicle when the compound of the disclosure is administered intravenously. Saline solutions and aqueous dextrose and glycerol solutions can also be employed as liquid vehicles, particularly for injectable solutions. Suitable pharmaceutical vehicles also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The pharmaceutical compositions may further contain one or more auxiliary substance, such as wetting or emulsifying agents, pH buffering agents, or adjuvants to enhance the effectiveness thereof.

In another embodiment, the compounds and/or compositions of the disclosure (radiolabeled antibodies) are formulated in accordance with routine procedures as a pharmaceutical composition adapted for intravenous administration to mammals, including humans. Typically, compounds and/or compositions of the disclosure for intravenous administration are solutions in sterile isotonic aqueous buffer. Where necessary, the compositions may also include a solubilizing agent. Compositions for intravenous administration may optionally include a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the compound of the disclosure is to be administered by infusion, it can be dispensed, for example, with an infusion bottle containing sterile pharmaceutical grade water or saline. Where the compound of the disclosure is administered by injection, an ampoule of sterile water for injection or saline can be provided so that the ingredients may be mixed prior to administration.

The amount of a compound of the disclosure that will be effective in the treatment of a particular disorder or condition disclosed herein will depend on the nature of the disorder or condition, and can be determined by standard clinical techniques. In addition, in vitro or in vivo assays may optionally be employed to help identify optimal dosage ranges. The precise dose to be employed in the compositions will also depend on the route of administration, and the seriousness of the disease or disorder, and should be decided according to the judgment of the practitioner and each patient's circumstances. In specific embodiments of the disclosure, the oral dose of at least one compound of the present disclosure is about 0.01 milligram to about 100 milligrams per kilogram body weight, or from about 0.1 milligram to about 50 milligrams per kilogram body weight, or from about 0.5 milligram to about 20 milligrams per kilogram body weight, or from about 1 milligram to about 10 milligrams per kilogram body weight.

Suitable dosage ranges for parenteral, for example, intravenous (IV) administration are 0.01 milligram to 100 milligrams radiolabeled antibody per kilogram body weight, 0.1 milligram to 35 milligrams per kilogram body weight, and 1 milligram to 10 milligrams per kilogram body weight. In other embodiments, a composition of the disclosure for parenteral, for example, intravenous administration includes about 0.001 milligram to about 2000 milligrams of a compound of the disclosure, from about 0.01 milligram to about 1000 milligrams of a compound of the disclosure, from about 0.1 milligram to about 500 milligrams of a compound of the disclosure, or from about 1 milligram to about 200 milligrams of a compound of the disclosure.

The disclosure also provides pharmaceutical packs or kits comprising one or more containers filled with one or more of the radiolabeled antibodies or antibody fragments of the disclosure. Optionally associated with such container(s) can be a notice in the form prescribed by a governmental agency regulating the manufacture, use or sale of pharmaceuticals or biological products, which notice reflects approval by the agency of manufacture, use or sale for human administration.

A therapeutically effective amount of the radiolabeled antibodies or antibody fragments in the composition should be administered, in which “a therapeutically effective amount” is defined as an amount that is sufficient to produce a desired prophylactic, therapeutic or ameliorative response in a subject. The amount needed will vary depending upon the antibodies or antibody fragments used and the species and weight of the subject to be administered but may be ascertained using standard techniques.

### 4. Methods of Treatment

The present disclosure provides methods for treating CUB domain-containing protein 1 (CDCP1)-positive prostate cancer, comprising: administering to a subject in need thereof a therapeutic radioisotope-labeled antibody or antibody fragment that specifically binds CDCP1 ectodomain to treat the prostate cancer, wherein the therapeutic radioisotope is an alpha-emitter and/or a beta-minus-emitter. Additionally, the present disclosure provides kits for a treating CUB domain-containing protein 1 (CDCP1)-positive prostate cancer, comprising: a therapeutic radioisotope-labeled antibody or antibody fragment that specifically binds CDCP1 ectodomain; and instructions for use of the therapeutic radioisotope-labeled antibody or antibody fragment to treat the prostate cancer. In some embodiments, the therapeutic radioisotope is selected from the group consisting of ⁶⁷Cu, ⁹⁰Y, ¹³¹L ¹⁵³Sm, ¹⁷⁷Lu, ²¹¹At, ²¹²Pb, ²²³Ra, ²²⁵Ac, and ²²⁷Th. In exemplary embodiments, the therapeutic radioisotope is ¹⁷⁷Lu.

When expressed as units of weight, an effective amount of a therapeutic radioisotope-labeled antibody or antibody fragment for treating CDCP1-positive prostate cancer is in the range of about 0.1 μg to about 100 mg, preferably from about 1 μg to about 10 mg, preferably from about 10 μg to about 1 mg, or preferably from about 50 μg to about 500 μg. When expressed as units of radioactivity, an effective amount of a therapeutic radioisotope-labeled antibody or antibody fragment for treating CDCP1-positive prostate cancer is in the range of from about 1 μCi to about 10 mCi, or preferably from about 10 μCi to about 1 mCi, or preferably from about 50 μCi to about 500 μCi.

In some embodiments, the prostate cancer is wherein the prostate cancer is castration-resistant prostate cancer (CRPC). In some embodiments, the prostate cancer is metastatic prostate cancer (mPC). In some embodiments, the prostate cancer is metastatic castration-resistant prostate cancer (mCRPC). In some embodiments, the prostate cancer is an adenocarcinoma subtype. In other embodiments, the prostate cancer is a neuroendocrine subtype. In some embodiments, cells of the prostate cancer expresses little or no prostate-specific membrane antigen (PSMA). That is, in some embodiments, cells of the prostate cancer are PSMA-negative.

Further provided are methods of suppressing or inhibiting the growth of, or reducing the growth rate of, prostate cancer cells that express CDCP1, comprising contacting the cells with, or administering to the cells, a pharmaceutically effective amount of a composition comprising at least one of the radiolabeled anti-CDCP1 antibodies or antibody fragments of the present disclosure. In certain specific embodiments, the prostate cancer cells harbor a KRAS mutation.

Also provided herein is a method of treating a subject having a prostate cancer, e.g., a prostate cancer expressing CDCP1 protein, comprising administering to the subject a therapeutically effective amount of a radiolabeled anti-CDCP1 antibody or antibody fragment provided herein. Further provided herein is a method of reducing the likelihood of prostate cancer metastasis, comprising administering to a subject in need of such treatment a therapeutically effective amount of a composition comprising a radiolabeled anti-CDCP1 antibody or antibody fragment provided herein. In certain specific embodiments, the prostate cancer comprises a KRAS mutation.

In other embodiments, the antibodies or antibody fragments of the disclosure are administered to a patient, for example a human, as a preventative measure against diseases, including preventing the occurrence of a tumor or preventing the progression of a tumor.

The subject is preferably a human but can be another mammal. The methods of treatment or prophylaxis of prostate cancer comprise administering to a subject in need of treatment thereof a pharmaceutically effective amount of a composition comprising at least one of the radiolabeled anti-CDCP1 antibodies or antibody fragments provided herein.

As used herein and unless otherwise indicated, the terms “cancer” or “cancer cell” refer to abnormal cell growth or proliferation that may or may not include spontaneous or induced phenotypic changes. As used herein, “cancer” includes but is not limited to such abnormal conditions as hypertrophy, neoplasia, hyperplasia, benign and malignant cancer. As used herein, the term “tumor” is a general term that includes hypertrophies, neoplasias, hyperplasias, benign cancers and malignant cancers. Accordingly, certain embodiments of the present disclosure include but are not limited to treating a hypertrophy, a neoplasia, a hyperplasia, a benign or a malignant cancer in a subject. In additional embodiments, the present disclosure is directed to preventing or reducing the likelihood of metastasis and/or recurrence of a hypertrophy, a neoplasia, a hyperplasia, a benign or a malignant cancer within a subject comprising administering at least one compound of the present disclosure to the subject. For example, at least one compound of the present disclosure may be administered after tumor resection/removal/ablation, etc. to reduce the likelihood of recurrence of the tumor in the subject. In another example, at least one compound of the present disclosure may be administered to reduce the likelihood of metastasis of the tumor in the subject.

As used herein, “treatment” or “treating” refers to an amelioration of a disease or disorder, or at least one detectable symptom thereof, or amelioration of at least one measurable physical parameter, not necessarily discernible by the patient. “Treatment” or “treating” may also refer to inhibiting the progression of a disease or disorder, either physically, e.g., stabilization of a discernible symptom, physiologically, e.g., stabilization of a physical parameter, or both, or to delaying the onset of a disease or disorder.

As used herein, the term “prevent,” as it relates to tumors and/or abnormal cell growth, indicates that the antibodies or antibody fragments of the present disclosure is administered to a subject to at least partially inhibit or reduce the likelihood of growth, division, spread, or proliferation of cancer cells. Of course, the term “prevent” also encompasses prohibiting entirely the emergence of new tumors or any of the associated symptoms from detectably appearing. Thus, a subject may be “pretreated,” by administering the one or more the antibodies or antibody fragments of the present disclosure to prevent tumors from arising. The phrase “preventing the progression,” as it relates to tumors, is used to mean a procedure designed to at least partially inhibit the detectable appearance of one or more additional tumors or aberrant cell growth in a patient already exhibiting one or more symptoms of the presence of a tumor or aberrant cell growth, and is also used to mean at least partially prohibiting the already-present symptoms of cancer from worsening in the subject.

As used herein, the term “administer” and “administering” are used to mean introducing at least one of the antibodies or antibody fragments to the cells in close enough proximity that the antibodies or antibody fragments can exert an effect on the cells. For example, “administer,” in an in vivo setting refers to introducing the antibodies or antibody fragments to the subject in need of treatment or prophylactic treatment thereof. In an in vitro, e.g., cell culture, setting, the term “administer” can mean to introduce the antibodies or antibody fragments into the cell culture environment such that the antibodies or antibody fragments contact the cells. When administration is for the purpose of treatment of a subject in need of treatment thereof, the antibodies or antibody fragments can be provided at, or after the diagnosis of an abnormal cell growth, such as a tumor. The therapeutic administration of the antibodies or antibody fragments serves to inhibit cell growth of the tumor or abnormal cell growth.

### 5. Methods of Detection

The disclosure also provides methods for assessing CUB domain-containing protein 1 (CDCP1) expression in vivo, comprising: (a) administering a diagnostic radioisotope-labeled antibody or antibody fragment that specifically binds the CDCP1 ectodomain to a subject having prostate cancer; and (b) visualizing binding of the antibody or antibody fragment to cells of the subject by radionuclide imaging to assess CDC1 expression, wherein the diagnostic radioisotope is a positron-emitter. Additionally, the present disclosure provides kits for assessing CUB domain-containing protein 1 (CDCP1) expression in vivo, comprising: a diagnostic radioisotope-labeled antibody or antibody fragment that specifically binds CDCP1 ectodomain; and instructions for use of the diagnostic radioisotope-labeled antibody or antibody fragment to visualize binding of the diagnostic radioisotope-labeled antibody or antibody fragment to cells of the subject by radionuclide imaging. The positron-emitter may be a beta-plus-emitter and/or a gamma-emitter. In some embodiments, the diagnostic radioisotope is selected from the group consisting of ¹⁸F, ⁴⁴Sc ⁶⁴Cu, ⁶⁸Ga, ⁸⁹Zr, and ¹²⁴I. In exemplary embodiments, the diagnostic radioisotope is ⁸⁹Zr. In some embodiments, the radionuclide imagining comprises positron emission tomography (PET). In some embodiments, the radionuclide imaging comprises single-photon emission computed tomography (SPECT).

When expressed as units of weight, an effective amount of a diagnostic radioisotope-labeled antibody or antibody fragment for assessing CDCP1 expression in vivo is in the range of about 1 μg to about 10 mg, preferably from about 10 μg to about 1 mg, or preferably from about 50 μg to about 500 μg. When expressed as units of radioactivity, an effective amount of a diagnostic radioisotope-labeled antibody or antibody fragment is in the range of from about 1 μCi to about 10 mCi, or preferably from about 10 μCi to about 1 mCi, or preferably from about 50 μCi to about 500 μCi.

In some embodiments, the prostate cancer is wherein the prostate cancer is castration-resistant prostate cancer (CRPC). In some embodiments, the prostate cancer is metastatic prostate cancer (mPC). In some embodiments, the prostate cancer is metastatic castration-resistant prostate cancer (mCRPC). In some embodiments, the prostate cancer is an adenocarcinoma subtype. In other embodiments, the prostate cancer is a neuroendocrine subtype. In some embodiments, cells of the prostate cancer expresses little or no prostate-specific membrane antigen (PSMA). That is, in some embodiments, cells of the prostate cancer are PSMA-negative.

### Enumerated Embodiments

1. A method for treating CUB domain-containing protein 1 (CDCP1)-positive prostate cancer, comprising:
administering to a subject in need thereof a therapeutic radioisotope-labeled antibody or antibody fragment that specifically binds CDCP1 ectodomain to treat the prostate cancer, wherein the therapeutic radioisotope is an alpha-emitter and/or a beta-minus-emitter.

2. The method of embodiment 1, wherein the antibody or antibody fragment comprises a light chain CDR1 comprising SEQ ID NO:11, a light chain CDR2 comprising SEQ ID NO:13, and:
(a) a light chain CDR3 comprising SEQ ID NO:20, a heavy chain CDR1 comprising SEQ ID NO:17, a heavy chain CDR2 comprising SEQ ID NO:18, and a heavy chain CDR3 comprising SEQ ID NO:19;
(b) a light chain CDR3 comprising SEQ ID NO:24, a heavy chain CDR1 comprising SEQ ID NO:21, a heavy chain CDR2 comprising SEQ ID NO:22, and a heavy chain CDR3 comprising SEQ ID NO:23;
(c) a light chain CDR3 comprising SEQ ID NO:28, a heavy chain CDR1 comprising SEQ ID NO:25, a heavy chain CDR2 comprising SEQ ID NO:26, and a heavy chain CDR3 comprising SEQ ID NO:27;
(d) a light chain CDR3 comprising SEQ ID NO:32, a heavy chain CDR1 comprising SEQ ID NO:29, a heavy chain CDR2 comprising SEQ ID NO:30, and a heavy chain CDR3 comprising SEQ ID NO:31;
(e) a light chain CDR3 comprising SEQ ID NO:36, a heavy chain CDR1 comprising SEQ ID NO:33, a heavy chain CDR2 comprising SEQ ID NO:34, and a heavy chain CDR3 comprising SEQ ID NO:35;
(f) a light chain CDR3 comprising SEQ ID NO:40, a heavy chain CDR1 comprising SEQ ID NO:37, a heavy chain CDR2 comprising SEQ ID NO:38, and a heavy chain CDR3 comprising SEQ ID NO:39;
(g) a light chain CDR3 comprising SEQ ID NO:44, a heavy chain CDR1 comprising SEQ ID NO:41, a heavy chain CDR2 comprising SEQ ID NO:42, and a heavy chain CDR3 comprising SEQ ID NO:43;
(h) a light chain CDR3 comprising SEQ ID NO:48, a heavy chain CDR1 comprising SEQ ID NO:45, a heavy chain CDR2 comprising SEQ ID NO:46, and a heavy chain CDR3 comprising SEQ ID NO:47;
(i) a light chain CDR3 comprising SEQ ID NO:52, a heavy chain CDR1 comprising SEQ ID NO:49, a heavy chain CDR2 comprising SEQ ID NO:50, and a heavy chain CDR3 comprising SEQ ID NO:51; or
(j) a light chain CDR3 comprising SEQ ID NO:56, a heavy chain CDR1 comprising SEQ ID NO:53, a heavy chain CDR2 comprising SEQ ID NO:54, and a heavy chain CDR3 comprising SEQ ID NO:55.

3. The method of embodiment 1, wherein the antibody or antibody fragment comprises light chain CDRs of SEQ ID NO:11, SEQ ID NO:13 and SEQ ID NO:20, and heavy chain CDRs of SEQ ID NO:17, SEQ ID NO:18, and SEQ ID NO:19.

4. The method of embodiment 1, wherein the antibody or antibody fragment comprises a heavy chain variable domain (VH) and a light chain variable domain (VL), and the VH comprises an amino acid sequence having at least 95% sequence identity to SEQ ID NO:57, and the VL comprises an amino acid sequence having at least 95% sequence identity to SEQ ID NO:58.

5. The method of embodiment 1, wherein the antibody or antibody fragment comprises a heavy chain variable domain (VH) and a light chain variable domain (VL), and the VH comprises the amino acid sequence of SEQ ID NO:57, and the VL comprises the amino acid sequence of SEQ ID NO:58.

6. The method of any one of embodiments 1-5, wherein the antibody or antibody fragment is a recombinant IgG₁ antibody.

7. The method of any one of embodiments 1-6, wherein the therapeutic radioisotope is selected from the group consisting of ⁶⁷Cu, ⁹⁰Y, ¹³¹L ¹⁵³Sm, ¹⁷⁷Lu, ²¹¹At, ²¹²Pb, ²²³Ra, ²²⁵Ac, and ²²⁷Th.

8. The method of any one of embodiments 1-6, wherein the therapeutic radioisotope is ¹⁷⁷Lu or ²²⁵Ac.

9. The method of any one of embodiments 1-6, wherein cells of the prostate cancer are prostate-specific membrane antigen (PSMA) negative.

10. The method of any one of embodiments 1-9, wherein the prostate cancer is castration-resistant prostate cancer (CRPC), optionally wherein the prostate cancer is metastatic castration-resistant prostate cancer (mCRPC).

11. The method of any one of embodiments 1-10, wherein the prostate cancer is an adenocarcinoma subtype or a neuroendocrine subtype.

12. The method of any one of embodiments 1-11, further comprising prior to administering the therapeutic radioisotope-labeled antibody or antibody fragment: determining the prostate cancer is CDCP1-positive by immunohistochemistry analysis of a prostate cancer biopsy obtained from the subject.

13. The method of any one of embodiments 1-12, further comprising prior to administering the therapeutic radioisotope-labeled antibody or antibody fragment:
determining the prostate cancer is CDCP1-positive by radionuclide imaging following administering a diagnostic radioisotope-labeled antibody or antibody fragment that specifically binds the CDCP1 ectodomain to the subject, wherein the diagnostic radioisotope is a positron-emitter.

14. The method of embodiment 13, wherein the diagnostic radioisotope is selected from the group consisting of ¹⁸F, ⁴⁴Sc 64Cu, ⁶⁸Ga, ⁸⁹Zr, and ¹²⁴I.

15. The method of embodiment 13, wherein the diagnostic radioisotope is ⁸⁹Zr.

16. The method of any one of embodiments 13-15, wherein the radionuclide imagining comprises positron emission tomography (PET) or single-photon emission computed tomography (SPECT).

17. The method of any one of embodiments 1-16, wherein one or both of the therapeutic radioisotope-labeled antibody or antibody fragment and the diagnostic radioisotope-labeled antibody or fragment are administered intravenously.

18. The method of any one of embodiments 1-16, wherein the prostate cancer is an adenocarcinoma or a small-cell neuroendocrine carcinoma (SCNC).

19. A method for assessing CUB domain-containing protein 1 (CDCP1) expression, comprising:
(a) administering a diagnostic radioisotope-labeled antibody or antibody fragment that specifically binds the CDCP1 ectodomain to a subject having prostate cancer; and
(b) visualizing binding of the antibody or antibody fragment to cells of the subject by radionuclide imaging to assess CDC1 expression, wherein the diagnostic radioisotope is a positron inhibitor.

20. The method of embodiment 19, wherein the antibody or antibody fragment comprises a light chain CDR1 comprising SEQ ID NO:11, a light chain CDR2 comprising SEQ ID NO:13, and:
(a) a light chain CDR3 comprising SEQ ID NO:20, a heavy chain CDR1 comprising SEQ ID NO:17, a heavy chain CDR2 comprising SEQ ID NO:18, and a heavy chain CDR3 comprising SEQ ID NO:19;
(b) a light chain CDR3 comprising SEQ ID NO:24, a heavy chain CDR1 comprising SEQ ID NO:21, a heavy chain CDR2 comprising SEQ ID NO:22, and a heavy chain CDR3 comprising SEQ ID NO:23;
(c) a light chain CDR3 comprising SEQ ID NO:28, a heavy chain CDR1 comprising SEQ ID NO:25, a heavy chain CDR2 comprising SEQ ID NO:26, and a heavy chain CDR3 comprising SEQ ID NO:27;
(d) a light chain CDR3 comprising SEQ ID NO:32, a heavy chain CDR1 comprising SEQ ID NO:29, a heavy chain CDR2 comprising SEQ ID NO:30, and a heavy chain CDR3 comprising SEQ ID NO:31;
(e) a light chain CDR3 comprising SEQ ID NO:36, a heavy chain CDR1 comprising SEQ ID NO:33, a heavy chain CDR2 comprising SEQ ID NO:34, and a heavy chain CDR3 comprising SEQ ID NO:35;
(f) a light chain CDR3 comprising SEQ ID NO:40, a heavy chain CDR1 comprising SEQ ID NO:37, a heavy chain CDR2 comprising SEQ ID NO:38, and a heavy chain CDR3 comprising SEQ ID NO:39;
(g) a light chain CDR3 comprising SEQ ID NO:44, a heavy chain CDR1 comprising SEQ ID NO:41, a heavy chain CDR2 comprising SEQ ID NO:42, and a heavy chain CDR3 comprising SEQ ID NO:43;
(h) a light chain CDR3 comprising SEQ ID NO:48, a heavy chain CDR1 comprising SEQ ID NO:45, a heavy chain CDR2 comprising SEQ ID NO:46, and a heavy chain CDR3 comprising SEQ ID NO:47;
(i) a light chain CDR3 comprising SEQ ID NO:52, a heavy chain CDR1 comprising SEQ ID NO:49, a heavy chain CDR2 comprising SEQ ID NO:50, and a heavy chain CDR3 comprising SEQ ID NO:51; or
(j) a light chain CDR3 comprising SEQ ID NO:56, a heavy chain CDR1 comprising SEQ ID NO:53, a heavy chain CDR2 comprising SEQ ID NO:54, and a heavy chain CDR3 comprising SEQ ID NO:55.

21. The method of embodiment 19, wherein the antibody or antibody fragment comprises light chain CDRs of SEQ ID NO:11, SEQ ID NO:13 and SEQ ID NO:20, and heavy chain CDRs of SEQ ID NO:17, SEQ ID NO:18, and SEQ ID NO:19.

22. The method of embodiment 19, wherein the antibody or antibody fragment comprises a heavy chain variable domain (VH) and a light chain variable domain (VL), and the VH comprises an amino acid sequence having at least 95% sequence identity to SEQ ID NO:57, and the VL comprises an amino acid sequence having at least 95% sequence identity to SEQ ID NO:58.

23. The method of embodiment 19, wherein the antibody or antibody fragment comprises a heavy chain variable domain (VH) and a light chain variable domain (VL), and the VH comprises the amino acid sequence of SEQ ID NO:57, and the VL comprises the amino acid sequence of SEQ ID NO:58.

24. The method of any one of embodiments 19-23, wherein the diagnostic radioisotope is selected from the group consisting of ¹⁸F, 44Sc ⁶⁴Cu, ⁶⁸Ga, ⁸⁹Zr, and ¹²⁴I.

25. The method of any one of embodiments 19-23, wherein the diagnostic radioisotope is ⁸⁹Zr.

26. The method of any one of embodiments 19-25, wherein the radionuclide imagining comprises positron emission tomography (PET) or single-photon emission computed tomography (SPECT).

27. The method of any one of embodiments 19-23, wherein cells of the prostate cancer are prostate-specific membrane antigen (PSMA) negative.

28. The method of any one of embodiments 19-27, wherein the prostate cancer is castration-resistant prostate cancer (CRPC), optionally wherein the prostate cancer is metastatic castration-resistant prostate cancer (mCRPC).

29. The method of any one of embodiments 19-28, wherein the prostate cancer is an adenocarcinoma subtype or a neuroendocrine subtype.

30. The method of any one of embodiments 19-29, further comprising determining the subject has a CUB domain-containing protein 1 (CDCP1)-positive prostate cancer, and treating the CDCP1-positive prostate cancer according to the method of any one of embodiments 1-8.

### EXAMPLES

The examples presented herein are meant for illustrative purposes and are not intended to limit the full scope of the disclosure. The attached figures are meant to be integral parts of the specification of the disclosure.

Abbreviations: AR (androgen receptor); ARC (antibody radionuclide conjugate); CDCP1 (CUB domain-containing protein); CDR (complementarity determining region); CRPC (castration-resistant prostate cancer); ECD (extracellular domain); ELISA (enzyme-linked immunosorbent assay); Fab (antigen-binding antibody fragment); FACS (fluorescent activated cell sorting); FR (framework region); MFI (mean fluorescence intensity); MMAF (monomethyl auristatin F); mCRPC (metastatic CRPC); PDX (patient-derived xenograft); PET (positron emission tomography); PSMA (prostate-specific membrane antigen); SILAC (stable isotope labeling with amino acids in cell culture); SCNC (small-cell neuroendocrine carcinoma); SPECT (single-photon emission computed tomography); TRT (targeted radiotherapy); VH or V_{H} (heavy chain variable domain); VL or V_{L} (light chain variable domain); and WT (wild type).

### Example 1: Targeted Radioligand Therapy for Prostate Cancer Through Use of an Anti-CUB Domain Containing Protein 1 (CDCP1) Antibody

CDCP1 can be used as a target for targeted radioligand therapy for prostate cancer.

### Materials and Methods

General Methods. All materials and chemicals were purchased from commercial vendors and used without further processing/purification. DU145, 22RV1, C4-2B, and PC3 cell lines were obtained from American Type Tissue Collection (ATCC) and cultured according to manufacturer's recommendations. Cellular identity was authenticated by visually inspecting morphology and probing for signature expression markers on immunoblot. Mycoplasma contamination was tested within the first two passages after thawing cryostocks with the MycoAlert kit (Lonza). All cells were studied between passages 5 to 25. The recombinant monoclonal antibody 4A06 was expressed and purified in the IgG1 format as previously described (Martinko et al., eLIFE, 7:e31098, 2018). 4A06 (also referred to herein as CDCP1-002) binds to an extracellular epitope contained on both full length and cleaved human CDCP1. p-SCN-Bn-Deferoxamine (B-705) and p-SCN-Bn-DOTA (B-205) were purchased from Macrocyclics (Plano, TX). ⁸⁹Zr-oxalate was obtained from 3D Imaging, LLC (Maumelle, AR). ¹⁷⁷LuCl₃ was obtained from Oak Ridge National Laboratory. Iodine-125 was obtained from Perkin Elmer. ⁶⁸Ga-PSMA 11 was prepared by the radiopharmacy at UCSF according to previously reported protocol (Hope et al., J Nucl Med, 58(1):81-4, 2017). LTL xenograft samples were acquired form Living Tumor Laboratory (Vancouver, BC) and the LuCaP xenograft series was provided by Dr. Eva Corey at University of Washington. Table 1-1 lists the human PDX and human cell lines used in this study.

TABLE 1-1
Summary of Features of Prostate Cancer Models

| Name | Type | Histology | AR status | PSMA status |
|---|---|---|---|---|
| LuCaP 70 CR | PDX | Adenocarcinoma | Positive | Positive |
| LuCaP 77 CR | PDX | Adenocarcinoma | Positive | Positive |
| LTL313 HR | PDX | Adenocarcinoma | Positive | Positive |
| LTL331 | PDX | Adenocarcinoma | Positive | Positive |
| LTL484 | PDX | Adenocarcinoma | Positive | Weakly positive |
| LuCaP 93-007 | PDX | Neuroendocrine | Negative | Negative |
| LuCaP 145.1 | PDX | Neuroendocrine | Negative | Negative |
| LTL352 | PDX | Neuroendocrine | Negative | Negative |
| LTL610 | PDX | Neuroendocrine | Negative | Negative |
| LTL331R | PDX | Neuroendocrine | Negative | Negative |
| LTL370 | PDX | Neuroendocrine | Negative | Negative |
| LTL545 | PDX | Neuroendocrine | Negative | Negative |
| PC3 | cell line | Indeterminate | Negative | Negative |
| DU145 | cell line | Indeterminate | Negative | Negative |
| LNCaP-AR | cell line | Adenocarcinoma | Positive | Positive |
| C4-2B | cell line | Adenocarcinoma | Positive | Positive |
| 22Rv1 | cell line | Adenocarcinoma | Positive | Weakly positive |
| CWR22Pc | cell line | Adenocarcinoma | Positive | Weakly positive |

Analysis of Patient Biopsies. Written informed consent was obtained prior to collecting patient biopsies. The sample collection and analysis were performed in accordance with the ethical guidelines stipulated in the Declaration of Helsinki, and the study was review and approved by the Institutional Review Board at UCSF prior to its start. Recently acquired metastatic biopsies from mCRPC patients clinically annotated with survival data was used for analysis (Quigley et al., Cell, 175(3):889, 2018; Aggarwal et al., Mol Cancer Res, 17(6):1235-40, 2019). Patient samples were obtained using image-guided core needle biopsy of metastatic lesion in the bone or soft tissue. Laser capture microdissection was used to isolate samples enriched for cancer, with cores freshly frozen for RNA sequencing, and separate core formalin-fixed and paraffin embedded for immunohistochemical analysis.

SCNC status was determined via unsupervised hierarchical clustering using a previously validated gene signature and confirmed via evaluation by three experienced pathologists blinded to the clinical and genomic features for determination of consensus pathologic subclassification (Aggarwal et al., Mol Cancer Res, 17(6):1235-40, 2019). The following list of genes was used for hierarchical clustering to determine SCNC status: AR, TMPRSS2, GATA2, HOXB13, KLK3, FOXA1, NKX3-1, CHGB, FOXA2, SOX2, SCG2, NKX2-1, REST, SPDEF, NOTCH2, NOTCH2NL, ASCL1, ETV1, ETV4, ETV5, RB1, CDKN2A, E2F1. Linear regression between CDCP1 and FOLH1 expression was performed in R (v4.0.2), with the resulting P value and Pearson correlation coefficient reported.

The needle biopsies were fixed in 10% formalin overnight, transferred to 70% ethanol the next day, and then processed for paraffin embedding and sectioning. Sections of formalin-fixed, paraffin embedded (FFPE) tissue were placed into the Ventana Discovery Ultra automated slide stainer. Antigen retrieval was performed using heat-inactivated antigen retrieval buffer (Tris-EDTA) according to the manufacturer instructions (Roche) and then stained with the following primary antibodies: rabbit polyclonal CDCP1 (Cell Signaling Technology, #4115S, 1:50), mouse monoclonal PSMA (Dako Agilent, M3620 clone 3E6, 1:100) or rabbit monoclonal CD3 (Ventana clone 2GV6, #790-4341, 1:100) for 32 minutes at 36° C. Secondary antibodies (Anti-Rabbit HQ, Anti-Mouse alk phos, HQ-HRP, Purple HRP and Yellow AP for dual chromogenic stains, all from Ventana) were incubated for 12 min each, and DAB was used for detection for single stains. Slides were counterstained with hematoxylin per standard protocol. H-scores for CDCP1 and PSMA membrane staining were assigned by two independent pathologists (1+, 2+ and 3+ multiplied by the percentage), and the average of the H-scores was calculated.

Flow Cytometry. Actively proliferating cells were lifted mechanically from a tissue culture plate and placed in the primary antibody solution diluted in PBS (4A06, 1:1,000). After a 30 min incubation, the cells were washed with PBS multiple times and placed in a fluorophore conjugated secondary antibody solution for 30 minutes on ice (1:500, 109-546-097, Jackson ImmunoResearch). Cells were collected and washed before being placed in PBS and passed through a cell strainer. Samples were taken to a flow machine (FACS CantoII). Data analysis was performed using FlowJo.

Immunoblot. Tumor samples were added to Pierce RIPA lysis buffer (89900, ThermoFisher Scientific) with Halt protease and phosphatase inhibitors (1861281, ThermoFisher Scientific). The samples were homogenized using a probe sonicator (Omni TH-01) and then centrifuged for 15 minutes at 15,000×g. 4× SDS-loading buffer was added to protein lysates and 15 μg of lysate were resolved on a 4-12% Bis Tris gel (NWO4120BOX, Invitrogen). Gels were transferred onto an Immobilon-P membrane (IPVH00010, Millipore). Membranes were blocked in 5% milk in TBST for 90 minutes before being placed in a primary antibody solution. Primary antibodies used were CDCP1 (4115, Cell Signaling, 1:1000), PSMA (12815, Cell Signaling, 1:1000), and B-Actin (A5441, Sigma-Aldrich, 1:5000). Membranes were then washed and incubated with a secondary antibody solution for 30 min at room temperature. Secondary antibodies used were goat anti-rabbit (65-6120, Invitrogen, 1:5000) and goat anti-rat (62-6520, Invitrogen, 1:5000). Proteins were detected using West Pico Chemiluminescent Substrate for 20 seconds (34578, ThermoFisher Scientific) and then exposed to film (30-507, Blue Devil). Each immunoblot was reproduced at least once with freshly harvested protein samples.

Saturation binding assays. To prepare ¹²⁵I-4A06, 6.0 μL of ¹²⁵I in 1M NaOH (˜340 uCi) was added into a vial containing 100.0 μL of HEPES buffer (0.5 M). This solution was transferred to an iodination tube (Peirce) and 4A06 antibody (300 μg) was then added. The tube was incubated for 10 min at room temperature, and ITLC showed 85.0% radiolabeling efficiency (solvent: 20 nM citric acid). ¹²⁵I-4A06 was purified using a G-25 column. The final yield of the purified ¹²⁵I-4A06 was 68.58% (specific activity=1.13 μCi/μg) and purity was >99%.

DU145 and PC3 (0.6×10⁶ cell/well) were seeded on 12-well plates using DMEM (10% FBS). The cells were washed with PBS for the saturation binding assay. Total binding of ¹²⁵I-4A06 was determined by adding it to cell suspensions at seven concentrations from 0.025 nM to 10 nM. Non-specific binding was determined by adding 1000× cold 4A06 to ¹²⁵I-4A06/cell mixtures at 0.025 nM, 0.3 nM, and 10 nM. In all cases, cells were incubated with ¹²⁵I-4A06 at room temperature for 1 hr, washed with PBS, and lysed by adding 1.0 M NaOH. The bound and unbound radioactive fractions were collected and measured on a Hidex Gamma counter (Turku, FI). Bmax was calculated using Prism v8.0.

Animal studies. All animal studies, including housing and welfare monitoring, were conducted in compliance with Institutional Animal Care and Use Committee at UCSF. Animal imaging studies involving patient derived xenografts (PDX) utilized eight to ten week old intact male NOD SCID gamma (NSG) mice from Charles River Laboratory. PDX lines were obtained from the Living Tumor Lab at the Vancouver Prostate Centre. For tumor imaging or treatment studies with mCRPC xenografts from cell line implants, four to six-week-old intact male athymic nu/nu mice (Charles River) were utilized. Mice were inoculated subcutaneously (˜1.5×10⁶ cells) in the flank with a slurry of cells in 1:1 mixture (v/v) of media (DMEM) and Matrigel (Corning). Xenografts were generally palpable within 3-4 weeks after injection. Tumor bearing mice received ˜300 μCi of ⁸⁹Zr-406 or ˜300 μCi of ⁶⁸Ga-PSMA 11 for imaging studies.

Mice bearing subcutaneous DU145 tumors received ¹⁷⁷Lu-4A06 (400 μCi) or vehicle (saline) via tail vein at day 0 and day 5 of the study period, which began approximately 14-21 days post tumor inoculation. Mice bearing subcutaneous C4-2B tumors received ¹⁷⁷Lu-4A06 (300 μCi) or vehicle (saline) via tail vein at day 0 and day 5 of the study period, which began approximately 14-21 days post tumor inoculation. Mice bearing subcutaneous LTL-545 tumors received ²²⁵Ac-4A06 (0.8 μCi) or vehicle (saline) via tail vein at day 0 and day 7 of the study period, which began approximately 14-21 days post tumor inoculation.

Mice were arranged in treatment arms using a simple randomization approach. Animals were weighed at the time of injection, and once weekly until the completion of the study. Tumor volume measurements were calculated with calipers. The study endpoints were death due to tumor volume >2000 mm³ or ≥20% loss in mouse body weight. The researcher performing the tumor volume and body weight measurements was blinded to the treatment arms.

Small animal PET/CT. 4A06 was functionalized with desferrioxamine (DFO) and subsequently radiolabeled with Zr-89 as previously described (Moroz et al., Clin Cancer Res, 26(14):3608-15, 2020). Tumor-bearing mice received ˜200 Ci of ⁸⁹Zr-4A06 or 0.8 μCi of ²²⁵Ac-4A06 in 100 μL saline solution volume intravenously using a custom mouse tail vein catheter with a 28-gauge needle and a 100-150 mm long polyethylene microtubing. Mice were imaged on a small animal PET/CT scanner (Inveon, Siemens Healthcare, Malvern, PA). Animals were typically scanned for 30 minutes for PET, and the CT acquisition was performed for 10 minutes.

The co-registration between PET and CT images was obtained using the rigid transformation matrix generated prior to the imaging data acquisition since the geometry between PET and CT remained constant for each of PET/CT scans using the combined PET/CT scanner. For microPET/CT data, PET images were reconstructed using the ordered subsets expectation maximization algorithm (OSEM) provided by the scanner manufacturer. The parameters for OSEM were 16 subsets and 4 iterations, and the resulting reconstructed image volume was in a matrix of 128×128×159 with a voxel size of 0.0776 mm×0.0775 mm×0.0796 mm. CT images for attenuation correction were reconstructed using a conebeam Feldkamp algorithm provided by the scanner manufacturer. The data were acquired using x-ray tube voltage of 80 kVp and current of 0.5 mA for 120 angular steps over 220 degrees, and 175 ms of exposure at each angular step. The reconstructed CT volume was in a matrix of 512×512×700 with a voxel size of 0.195 mm×0.195 mm×0.195 mm. The precalibrated scaling was used to convert the CT images to attenuation maps for correction in PET reconstruction.

For SUV computation, we used freeware software, Amide (amide.sourceforge.net), and used its automated SUV calculation tool by entering decay-corrected injected activity and the animal weight. For each volume of interest, a spherical VOI (2-3 mm diameter) was drawn, and SUV was calculated by VOI statistics.

Small animal SPECT/CT. 4A06 was functionalized with DOTA and radiolabeled with Lu-177 as previously described (Moroz et al., Clin Cancer Res, 26(14):3608-15, 2020). At 48 hours post injection of the second dose of ¹⁷⁷Lu-4A06 (i.e., day 7 overall of the antitumor assessment study), the mice were imaged under anesthesia using a small animal SPECT/CT (VECTor4CT, MILabs, Utrecht, The Netherlands). Animals were typically scanned for 40 minutes for SPECT, and the CT acquisition was performed for 10 minutes. The co-registered CT was used for photon attenuation correction in the SPECT reconstruction. For microSPECT/CT data, SPECT images were reconstructed using the similarity-regulated OSEM (SROSEM) provided by the scanner manufacturer. The parameters for SROSEM were 128 subsets and 10 iterations in a base voxel size of 1.2 mm. After reconstruction, a Gaussian postfilter with 1.5 mm full-width at half maximum was applied to suppress image noises. SPECT data were acquired using a multipinhole collimator (HE-GP-RM) with an aperture diameter of 3.6 mm that was designed for general purpose to scan both mice and rats with the axial field of view (FOV) of 18 mm and transverse FOV of 28 mm. Multiple bed positions during the data acquisition were used, controlled by the scanner to cover the whole mouse volume. Both photopeaks (171 keV and 245 keV) for Lu-177 with +/−10% energy window was used from the list mode data, and triple window based scatter correction was applied. After the SPECT reconstruction, the SPECT images were registered to the CT images, and attenuation correction using CT-based attenuation map was applied. CT data were acquired using x-ray tube voltage of 55 kVp and current of 0.19 mA for 480 angular steps over 360 degrees, and 75 ms of exposure at each angular step. CT images were reconstructed using the manufacturer provided conebeam Feldkamp algorithm.

Biodistribution studies. At a dedicated time after radiotracer injection, animals were euthanized by cervical dislocation. Blood was harvested via cardiac puncture. Tissues were removed, weighed and counted on a Hidex automatic gamma counter (Turku, Finland). The activity of the injected radiotracer was calculated and used to determine the total number of counts per minute by comparison with a standard of known activity. The data were background- and decay-corrected and expressed as the percentage of the injected dose/weight of the biospecimen in grams (% ID/g).

Digital autoradiography. Post mortem, tumors were harvested, transferred to sample boats, and immersed in Tissue-Plus OCT compound (Scigen, Gardena, CA). The tissues were snap frozen at −80° C. The tumor tissue was sectioned into 20 m slices using a microtome (Leica, Buffalo Grove, IL) and mounted on glass microscope slides. The slides were loaded onto an autoradiography cassette and exposed with a GE phosphor storage screen for 24-72 hours at −20° C. The film was developed and read on a Typhoon 9400 phosphorimager (Marlborough, MA). Images of whole sections were acquired on a VERSA automated slide scanner (Leica Biosystems, Wetzlar, Germany), equipped with an Andor Zyla 5.5 sCMOS camera (Andor Technologies, Belfast, UK). ImageScope software (Aperio Technologies, Vista, CA) is used for creating individual images. Photoshop CS6 software (Adobe Systems, McLean, VA) were used for montage and processing.

Statistical analysis. Binary comparisons between two treatment arms were made with an unpaired, two-tailed Student's t-test. Differences at the 95% confidence level (P<0.05) were considered to be statistically significant. Differences at the 95% confidence level were considered statistically significant. Unless otherwise stated, all data were expressed as mean±standard deviation. Overall survival was measured from the date of metastatic biopsy in the mCRPC patients. Kaplan-Meier product limit method and log-rank test were used to investigate the relationship between CDCP1 expression (transcripts per million, TPM) and overall survival, or CDCP1 expression, PTEN mutation status and overall survival. The patient cohort was dichotomized above and below median CDCP1 expression as well as broken into quartiles of CDCP1 expression for survival analyses.

### Results

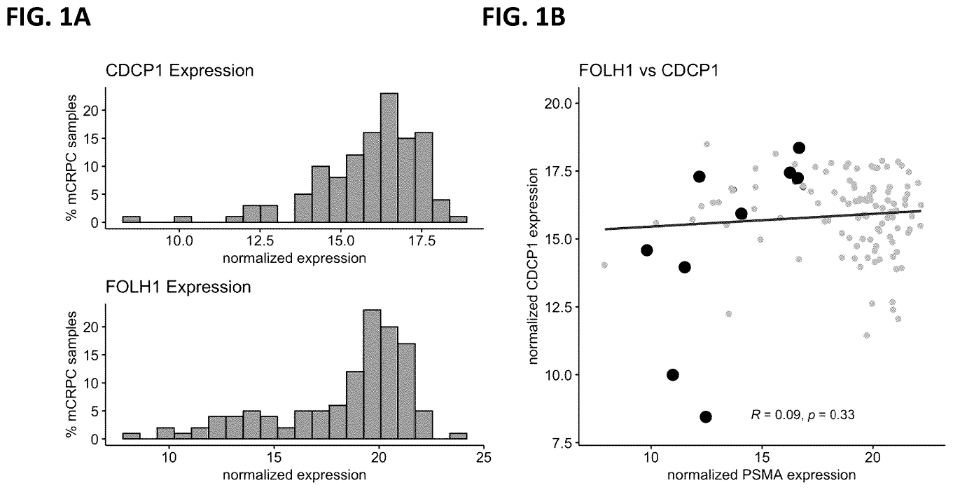

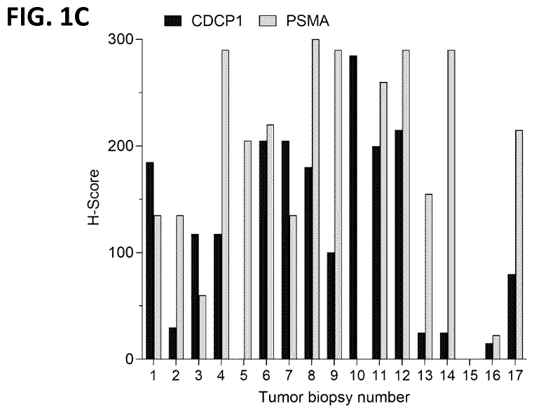

CDCP1 is expressed in mCRPC, including SCNC and adenocarcinoma with low levels of PSMA. CDCP1 expression was assessed in a RNA-seq data set of mCRPC biopsies (Aggarwal et al., Mol Cancer Res, 17(6):1235-1240, 2019). Unlike a previously reported expression analyses (Alajati et al., J Clin Invest, 130(5):2435-2450, 2020), the majority of the mCRPC biopsies from this data set were obtained post-abiraterone and/or enzalutamide from bone and soft tissue lesions, reflecting current treatment patterns. CDCP1 was expressed in 90% of mCRPC biopsies (107 of 119 samples, see FIG. 1A). Expression levels of CDCP1 and PTEN were compared. A Pearson analysis revealed that CDCP1 levels were modestly, but significantly, inversely correlated. CDCP1 mRNA levels were also compared between patients with wild type PTEN and patients with any type of PTEN mutation (i.e., deletion, inversion, break-end). CDCP1 was found to be slightly upregulated in PTEN-mutant patients, though the effect is not statistically significant (Table 1-2).

TABLE 1-2
Differential gene expression analysis between
patients with any type of PTEN mutation
(deletion, inversion, break-end) versus wild type PTEN

| | Log2 | Unadjusted | False |
|---|---|---|---|
| Gene | Fold Change | P value | discovery rate |
| PTEN | −1.44 | 2.1 × 10⁻⁶ | 0.04 |
| CDCP1 | 0.52 | 0.20 | 0.88 |

To understand if CDCP1 is expressed in tumors with low or undetectable PSMA, we next evaluated the distribution of CDCP1 versus FOLH1 (PSMA) expressing tumors. A Pearson analysis comparing the CDCP1 and FOLH1 expression per tumor showed that there was no significant correlation between the gene expression levels among the patients in the cohort (FIG. 1B). Of the 119 tumors, 14 (12%) were predicted to have low or absent FOLH1 expression and positive CDCP1 expression. In addition, 93 of 119 (78%) were predicted to be mutually positive, 3 of 119 (3%) were mutually negative, and 9 of 119 (8%) were predicted to be FOLH1 positive but CDCP1 negative.

We further evaluated the protein expression of CDCP1 in mCRPC biopsies. Two blinded pathologists reviewed the biopsies for CDCP1 and PSMA staining, and a mean H score was reported. Of 17 evaluable biopsies, 15 (85%) were positive for cell surface expression of CDCP1, with H-score range of 15-285 (FIG. 1C). Four samples (23%) had a higher mean H-score for CDCP1 compared to PSMA, including one example (biopsy #10) that overexpressed CDCP1 (H-score 285) but did express PSMA. Of note, one sample (biopsy #15) was negative on staining for both CDCP1 and PSMA.

The association between CDCP1 overexpression and poorer overall survival in mCRPC was assessed in this cohort. The results showed no significant association between CDCP1 expression and overall survival in dichotomized cohorts above and below median expression (log-rank p value=0.2) or by breaking into quartiles of CDCP1 expression (log-rank p-value=0.1). The results also showed no differences in survival when subdividing the patient cohort into subgroups based upon CDCP1 expression (above vs. below median) and PTEN mutation status (wild type vs. mutation, log-rank p-value=0.5).

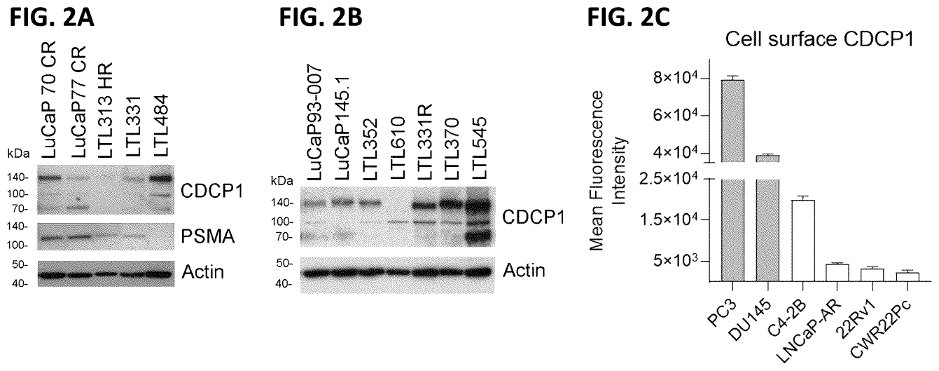

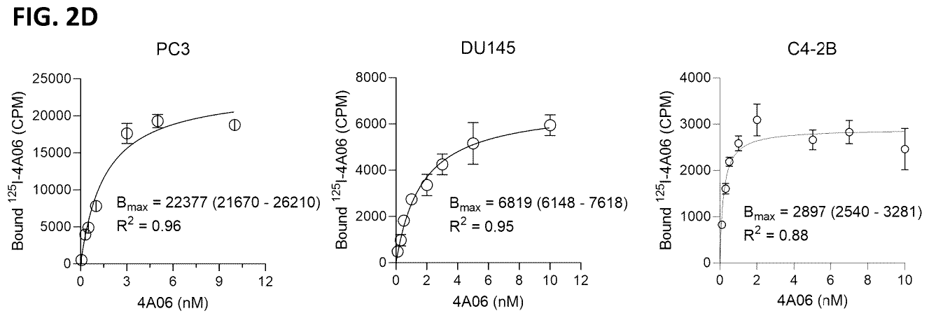

Since CDCP1 protein was already found to be expressed at high levels in the whole cell lysates of several human prostate cancer cell lines (Yang et al., Oncotarget, 6(41):43743-58, 2015), expression in human PDX whole cell lysates was evaluated. Full length and/or cleaved CDCP1 was expressed in four of five adenocarcinoma PDX models that we tested from the LuCaP and Living Tumor Laboratory series (FIG. 2A). The highest levels were observed in LuCaP70 CR and LTL-484, an AR-expressing adenocarcinoma sample with negligible PSMA expression. CDCP1 expression was detected in six of seven SCNC PDXs, with the highest expression observed in LTL-545 and LTL-370 (FIG. 2B). In line with some recent reports suggesting that proteolytically cleaved CDCP1 may contribute to early tumor development and metastatic potential (Casar et al., Oncogene, 33(2):255-68, 2014; Wright et al., Oncogene, 35(36):4762-72, 2016; Casar et al., Oncogene, 31(35):3924-38, 2012), this proteolytic isoform of CDCP1 was detected in 4 of the 12 PDX samples (LuCaP70 CR, LuCaP 77 CR, LTL484, LTL545).

Surface CDCP1 expression in prostate cancer cells using flow cytometry was assessed (FIG. 2C). The relative cell surface levels in six human prostate cancer cell lines (PC3>DU145>C4-2B>LNCaP-AR>CWR22Pc>22Rv1) approximated previously reported relative levels of total expression on immunoblot (Kryza et al., Theranostics, 10(9):4116-33, 2020). To quantify CDCP1 receptor number per cell, saturation binding assays were performed using 4A06, a monoclonal recombinant human antibody that recognizes a region of the CDCP1 ectodomain present in both full length and cleaved CDCP1 (Martinko et al., Elife, 7, 2018). As shown in FIG. 2D, PC3 cells had a Bₘₐₓ of 22,377 fmol/mg (˜1.2×10⁶ receptors per cell), DU145 had a Bₘₐₓ of 6819 fmol/mg (˜3.7×10⁵ receptors per cell) and C4-2B had a Bₘₐₓ of 2897 fmol/mg (˜1.5×10⁵ receptors per cell). These values equal or exceed receptor densities reported for PSMA in human prostate cancer cell lines like LNCaP and MDA PCa 2b (˜1×10⁶ receptors/cell) (Wang et al., Mol Cancer Ther, 10(9):1728-39, 2011).

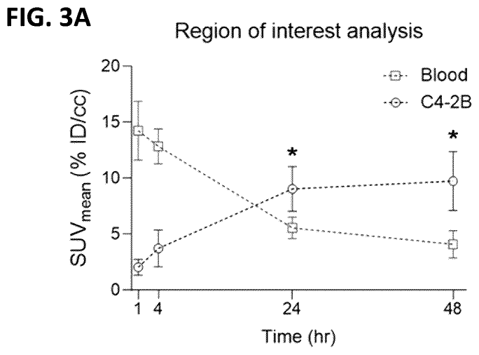

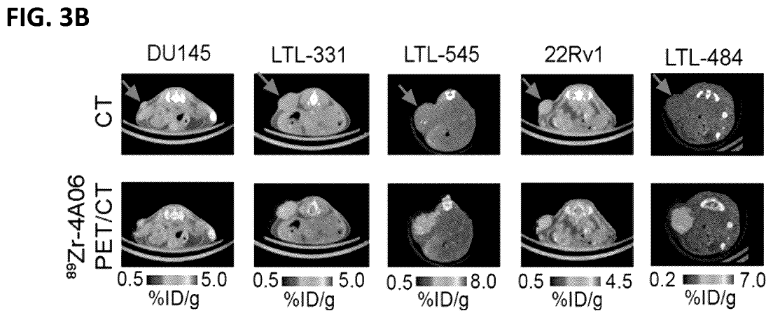

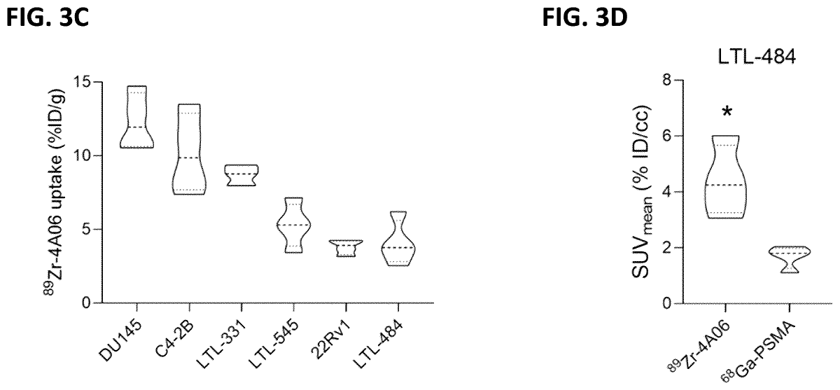

Tumor autonomous expression of CDCP1 in mCRPC is detectable with ⁸⁹Zr-4A06 PET/CT. CDCP1 expression in vivo was assessed using ⁸⁹Zr-labeled 4A06, an IgG1 monoclonal antibody that we previously developed that recognizes an epitope in the ectodomain found on both full-length and cleaved forms of CDCP1 (Martinko et al., Elife, 7, 2018). Tumor uptake of functionalized 4A06 was evaluated over time in intact male nu/nu mice bearing subcutaneous C4-2B tumors (n=4). As shown in FIG. 3A, tumoral uptake of ⁸⁹Zr-4A06 was significantly higher than blood levels at 24 hours post injection. Although tumoral uptake did not significantly change between 24- and 48-hours post injection, region of interest analysis suggested the tumor to blood ratio improved from 24 to 48 hours post injection (FIG. 3A).

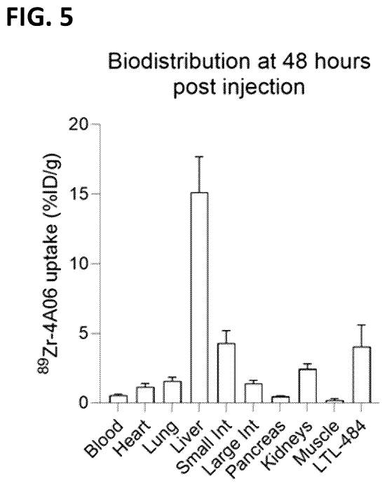

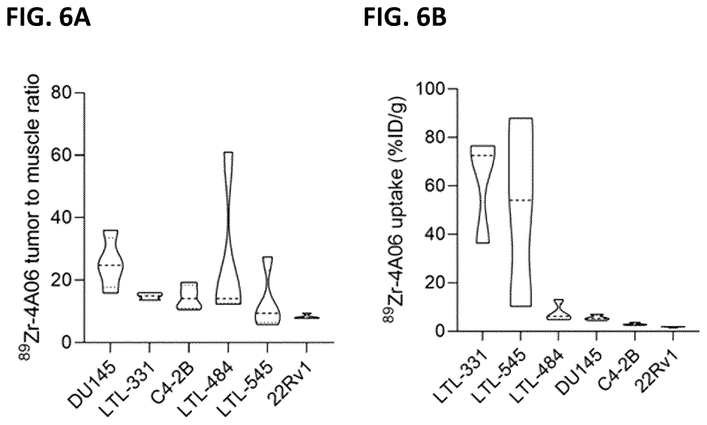

CDCP1 expression at 48 hours post injection was profiled in intact male nu/nu or NSG mice bearing DU145, 22Rv1, LTL-545, LTL-331, or LTL-484 tumor xenografts (n=4/tumor). These tumors represent AR-positive adenocarcinoma (22Rv1, LTL-331, LTL-484) and AR-null disease (DU145, LTL-545). ⁸⁹Zr-4A06 PET showed clear evidence of radiotracer accumulation in tumors above background levels in blood or skeletal muscle. Post mortem biodistribution measurements at 48 hours post injection showed that DU145 had the highest radiotracer uptake at 12.9±1.9% ID/g, and that the highest uptake was found in the liver (FIGS. 3B and 5). The tumor to muscle ratios were at least 7:1 and DU145 had the highest ratio at ˜25:1 (FIG. 6A). The tumor to blood ratios were at least 2:1 and the LTL-331 cohort had the highest mean ratio of ˜70:1 (FIG. 6B). The blood values in the NSG cohorts were lower than those observed in the nu/nu cohorts, which is likely due to high splenic uptake of the IgG as has been previously noted (Sharma et al., Cancer Res, 78(7):1820-32, 2018). Digital autoradiography was performed to assess radiotracer distribution within a tumor representing high (DU145) and comparatively lower (22Rv1) tracer uptake. Radiotracer distribution was qualitatively similar, and most abundant around the periphery of the tumor as expected for a large immunoglobulin like 4A06. Lastly, ⁶⁸Ga-PSMA 11 uptake was compared to ⁸⁹Zr-4A06 uptake in intact male mice bearing LTL-484 tumors (n=4). Consistent with the protein expression patterns, ⁸⁹Zr-4A06 uptake in tumors was significantly higher than ⁶⁸Ga-PSMA 11 (FIG. 3C).

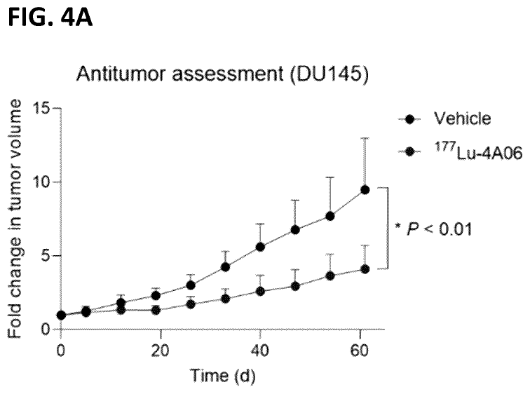

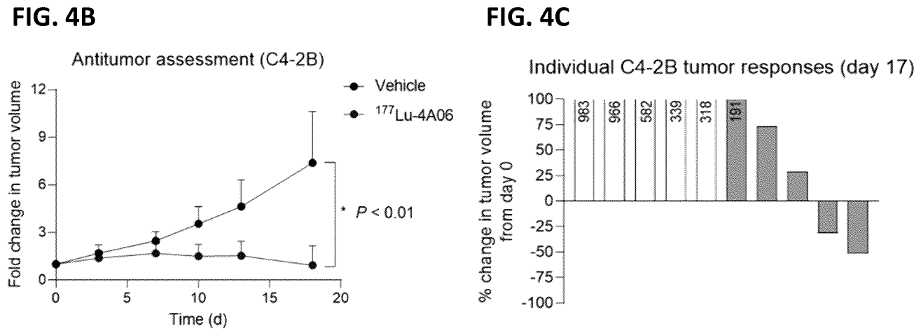

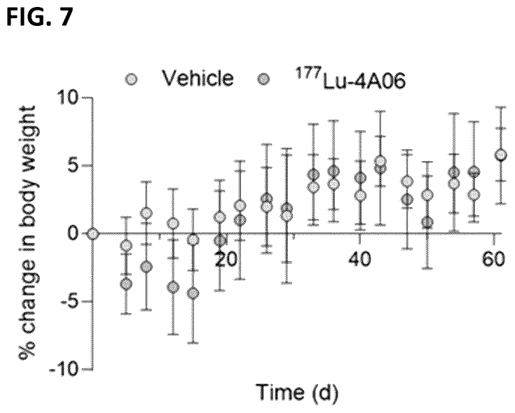

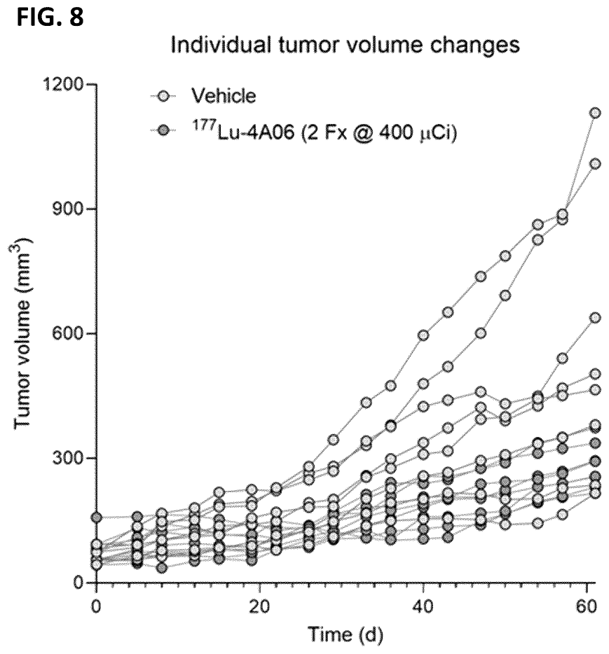

Radioligand therapy with ¹⁷⁷Lu-4A06 inhibits mCRPC tumor growth. Antitumor effects of ¹⁷⁷Lu-4A06 was tested in PSMA-null tumors. Intact male nu/nu mice bearing PSMA-negative DU145 tumors received ¹⁷⁷Lu-4A06 in two fractions of 400 μCi at day 0 and day 5 of the study. This dosing regimen was chosen as it was previously shown to be more efficacious that a single fraction of the same total radioactive dose (i.e., 800 μCi) (Moroz et al., Clin Cancer Res, 26(14):3608-15, 2020). SPECT/CT imaging showed effective tumor targeting at 72 hours after the first injection. ¹⁷⁷Lu-4A06 treatment significantly delayed tumor growth compared to vehicle with no evidence for unhealthy weight loss (FIGS. 4A, 7, and 8). To test if CDCP1 RLT is efficacious against AR-positive, PSMA-positive tumors, intact male nu/nu mice bearing subcutaneous C4-2B tumors were treated with ¹⁷⁷Lu-4A06 (2 fractions of 300 μCi at day 0 and 5) or vehicle. ¹⁷⁷Lu-4A06 significantly inhibited tumor growth in this model as well, and tumor regressions were observed in two of five mice (FIGS. 4B-C).

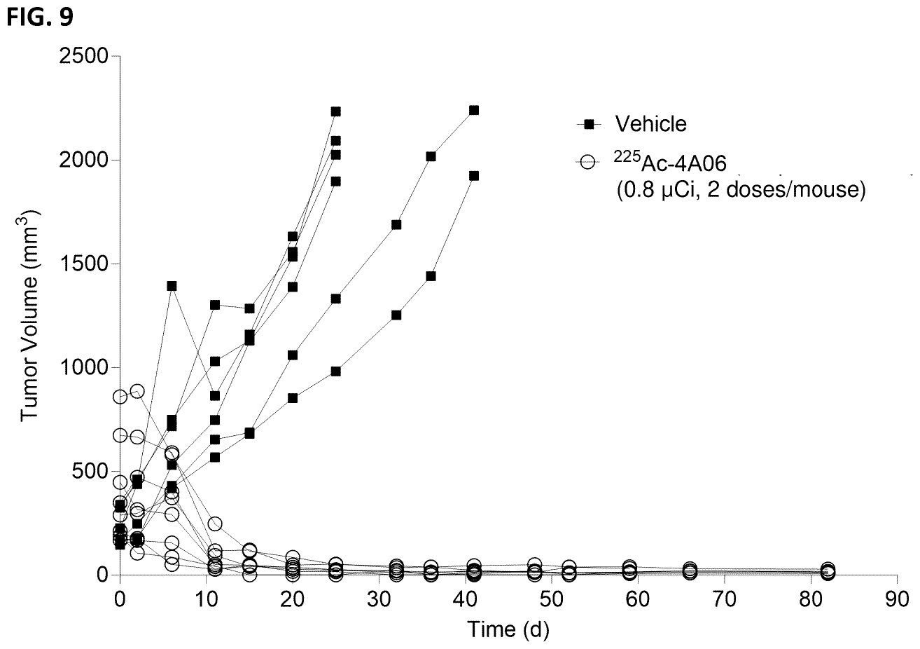

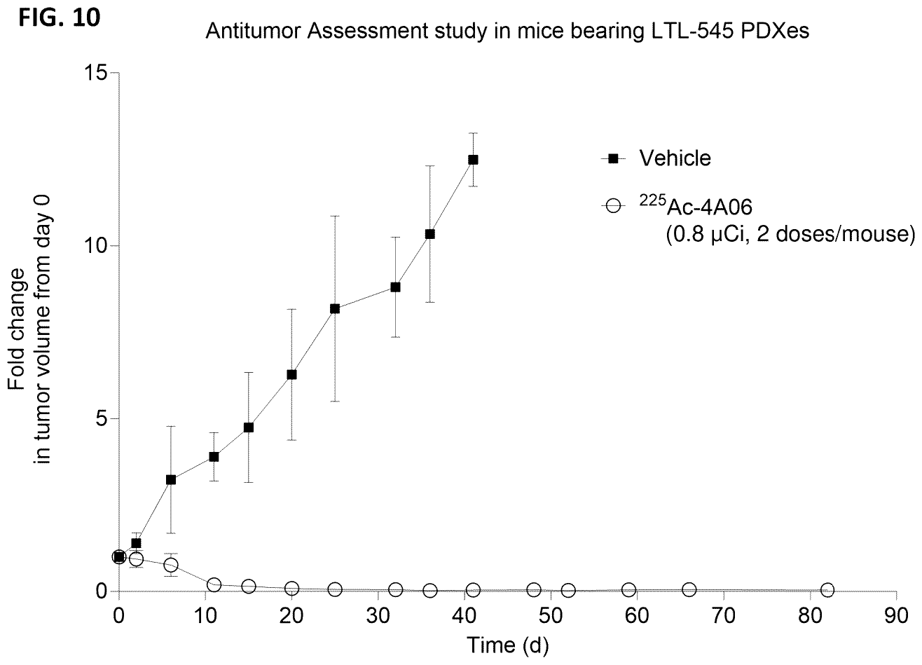

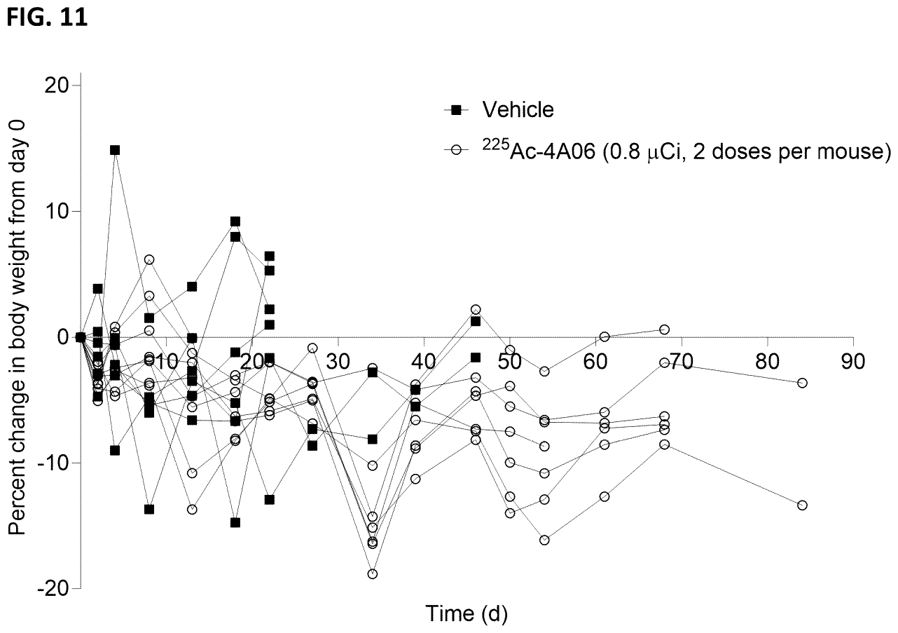

Radioligand therapy with ²²Ac-4A06 inhibits growth of androgen-independent, neuroendocrine carcinoma of the prostate. Antitumor effects of ²²⁵Ac-4A06 was tested in human LTL-545 prostate cancer xenografts. Intact male, athymic nu/nu mice (8/group) bearing LTL-545 tumors received ²²⁵Ac-4A06 in two fractions of 0.8 μCi at day 0 and day 7 of the study. Tumor volume was assessed every 4 days using calipers. ²²⁵Ac-4A06 treatment reduced tumor volume as shown in FIG. 9 and FIG. 10. Complete responses were observed in 6/8 mice in the ²²⁵Ac-4A06 treated group. No mice were euthanized due to unsafe weight loss (FIG. 11).

### CONCLUSIONS

We demonstrate for the first time that CDCP1 directed RLT is an appropriate therapeutic strategy for the treatment of mCRPC, including subtypes that cannot be addressed with PSMA-directed RLT or bone seeking radionuclides. This finding was enabled by expression analysis in mCRPC biopsies, which revealed that CDCP1 was expressed at the cell surface in over 60% of mCRPC biopsies, including in adenocarcinomas with negligible PSMA expression. Full length and/or cleaved CDCP1 was expressed in the majority of PSMA-null SCNC PDX models and in the majority of adenocarcinoma PDX models, including LTL-484 which has low PSMA expression. Our study demonstrates the feasibility of detecting CDCP1 expression on prostate cancer tumors in vivo using ⁸⁹Zr-4A06 PET. Moreover, we showed that ¹⁷⁷Lu-4A06 significantly suppressed the growth of PSMA-null DU145 tumors and induced tumor regression in mice bearing C4-2B tumors. In addition, we showed that ²²⁵Ac-4A06 induced tumor regression in mice bearing LTL-545 tumors.

As CDCP1 is cleaved on the cell surface by proteases like matriptase, an antibody like 4A06 that recognizes an epitope on the N-terminus in both full length and cleaved CDCP1 could be viewed as suboptimal for drug development. Indeed, one might expect that some portion of ⁸⁹Zr-4A06 would remain in circulation bound to a shed CDCP1 N-terminal fragment. However, we do not observe unusually high levels of ⁸⁹Zr-4A06 compared to other radiolabeled CDCP1 antibodies. Mechanistically, this is thought to be due at least in part because the cleaved N-terminal fragment remains tightly bound to the C-terminal fragment of the CDCP1 ectodomain (Lim et al., J Clin Invest, 132(4):e154604, 2022). Thus, co-targeting full length and cleaved CDCP1 is contemplated to be a desirable approach for targeted radiotherapy for prostate cancer.

We claim:

1. A method for treating CUB domain-containing protein 1 (CDCP1)-positive prostate cancer, comprising:
administering to a subject in need thereof a therapeutic radioisotope-labeled antibody or antibody fragment that specifically binds CDCP1 ectodomain to treat the prostate cancer, wherein the therapeutic radioisotope is an alpha-emitter and/or a beta-minus-emitter.

2. The method of claim 1, wherein the antibody or antibody fragment comprises a light chain CDR1 comprising SEQ ID NO:11, a light chain CDR2 comprising SEQ ID NO:13, and:
(a) a light chain CDR3 comprising SEQ ID NO:20, a heavy chain CDR1 comprising SEQ ID NO:17, a heavy chain CDR2 comprising SEQ ID NO:18, and a heavy chain CDR3 comprising SEQ ID NO:19;
(b) a light chain CDR3 comprising SEQ ID NO:24, a heavy chain CDR1 comprising SEQ ID NO:21, a heavy chain CDR2 comprising SEQ ID NO:22, and a heavy chain CDR3 comprising SEQ ID NO:23;
(c) a light chain CDR3 comprising SEQ ID NO:28, a heavy chain CDR1 comprising SEQ ID NO:25, a heavy chain CDR2 comprising SEQ ID NO:26, and a heavy chain CDR3 comprising SEQ ID NO:27;
(d) a light chain CDR3 comprising SEQ ID NO:32, a heavy chain CDR1 comprising SEQ ID NO:29, a heavy chain CDR2 comprising SEQ ID NO:30, and a heavy chain CDR3 comprising SEQ ID NO:31;
(e) a light chain CDR3 comprising SEQ ID NO:36, a heavy chain CDR1 comprising SEQ ID NO:33, a heavy chain CDR2 comprising SEQ ID NO:34, and a heavy chain CDR3 comprising SEQ ID NO:35;
(f) a light chain CDR3 comprising SEQ ID NO:40, a heavy chain CDR1 comprising SEQ ID NO:37, a heavy chain CDR2 comprising SEQ ID NO:38, and a heavy chain CDR3 comprising SEQ ID NO:39;
(g) a light chain CDR3 comprising SEQ ID NO:44, a heavy chain CDR1 comprising SEQ ID NO:41, a heavy chain CDR2 comprising SEQ ID NO:42, and a heavy chain CDR3 comprising SEQ ID NO:43;
(h) a light chain CDR3 comprising SEQ ID NO:48, a heavy chain CDR1 comprising SEQ ID NO:45, a heavy chain CDR2 comprising SEQ ID NO:46, and a heavy chain CDR3 comprising SEQ ID NO:47;
(i) a light chain CDR3 comprising SEQ ID NO:52, a heavy chain CDR1 comprising SEQ ID NO:49, a heavy chain CDR2 comprising SEQ ID NO:50, and a heavy chain CDR3 comprising SEQ ID NO:51; or
(j) a light chain CDR3 comprising SEQ ID NO:56, a heavy chain CDR1 comprising SEQ ID NO:53, a heavy chain CDR2 comprising SEQ ID NO:54, and a heavy chain CDR3 comprising SEQ ID NO:55.

3. The method of claim 1, wherein the antibody or antibody fragment comprises light chain CDRs of SEQ ID NO:11, SEQ ID NO:13 and SEQ ID NO:20, and heavy chain CDRs of SEQ ID NO:17, SEQ ID NO:18, and SEQ ID NO:19.

4. The method of claim 1, wherein the antibody or antibody fragment comprises a heavy chain variable domain (VH) and a light chain variable domain (VL), and the VH comprises an amino acid sequence having at least 95% sequence identity to SEQ ID NO:57, and the VL comprises an amino acid sequence having at least 95% sequence identity to SEQ ID NO:58.

5. The method of claim 1, wherein the antibody or antibody fragment comprises a heavy chain variable domain (VH) and a light chain variable domain (VL), and the VH comprises the amino acid sequence of SEQ ID NO:57, and the VL comprises the amino acid sequence of SEQ ID NO:58.

6. The method of claim 1, wherein the antibody or antibody fragment is a recombinant IgG₁ antibody.

7. The method of claim 1, wherein the therapeutic radioisotope is selected from the group consisting of ⁶⁷Cu, 90Y, ¹³¹L ¹⁵³Sm, ¹⁷⁷Lu, ²¹¹At, ²¹²Pb, ²²³Ra, ²²⁵Ac, and ²²⁷Th.

8. The method of claim 1, wherein the therapeutic radioisotope is ¹⁷⁷Lu or ²²⁵Ac.

9. The method of claim 1, wherein cells of the prostate cancer are prostate-specific membrane antigen (PSMA) negative.

10. The method of claim 1, wherein the prostate cancer is castration-resistant prostate cancer (CRPC), optionally wherein the prostate cancer is metastatic castration-resistant prostate cancer (mCRPC).

11. The method of claim 1, wherein the prostate cancer is an adenocarcinoma subtype or a neuroendocrine subtype.

12. The method of claim 1, further comprising prior to administering the therapeutic radioisotope-labeled antibody or antibody fragment:
determining the prostate cancer is CDCP1-positive by immunohistochemistry analysis of a prostate cancer biopsy obtained from the subject.

13. The method of 1, further comprising prior to administering the therapeutic radioisotope-labeled antibody or antibody fragment:
determining the prostate cancer is CDCP1-positive by radionuclide imaging following administering a diagnostic radioisotope-labeled antibody or antibody fragment that specifically binds the CDCP1 ectodomain to the subject, wherein the diagnostic radioisotope is a positron-emitter.

14. The method of claim 13, wherein the diagnostic radioisotope is selected from the group consisting of ¹⁸F, 44Sc ⁶⁴Cu, ⁶⁸Ga, ⁸⁹Zr, and ¹²⁴I.

15. The method of claim 13, wherein the diagnostic radioisotope is ⁸⁹Zr.

16. The method of claim 13, wherein the radionuclide imagining comprises positron emission tomography (PET) or single-photon emission computed tomography (SPECT).

17. The method of claim 13, wherein one or both of the therapeutic radioisotope-labeled antibody or antibody fragment and the diagnostic radioisotope-labeled antibody or fragment are administered intravenously.

18. The method of claim 1, wherein the prostate cancer is an adenocarcinoma or a small-cell neuroendocrine carcinoma (SCNC).

19. A method for assessing CUB domain-containing protein 1 (CDCP1) expression, comprising:
(a) administering a diagnostic radioisotope-labeled antibody or antibody fragment that specifically binds the CDCP1 ectodomain to a subject having prostate cancer; and
(b) visualizing binding of the antibody or antibody fragment to cells of the subject by radionuclide imaging to assess CDC1 expression, wherein
the diagnostic radioisotope is a positron inhibitor.

20. The method of claim 19, wherein the antibody or antibody fragment comprises a light chain CDR1 comprising SEQ ID NO:11, a light chain CDR2 comprising SEQ ID NO:13, and:
(a) a light chain CDR3 comprising SEQ ID NO:20, a heavy chain CDR1 comprising SEQ ID NO:17, a heavy chain CDR2 comprising SEQ ID NO:18, and a heavy chain CDR3 comprising SEQ ID NO:19;
(b) a light chain CDR3 comprising SEQ ID NO:24, a heavy chain CDR1 comprising SEQ ID NO:21, a heavy chain CDR2 comprising SEQ ID NO:22, and a heavy chain CDR3 comprising SEQ ID NO:23;
(c) a light chain CDR3 comprising SEQ ID NO:28, a heavy chain CDR1 comprising SEQ ID NO:25, a heavy chain CDR2 comprising SEQ ID NO:26, and a heavy chain CDR3 comprising SEQ ID NO:27;
(d) a light chain CDR3 comprising SEQ ID NO:32, a heavy chain CDR1 comprising SEQ ID NO:29, a heavy chain CDR2 comprising SEQ ID NO:30, and a heavy chain CDR3 comprising SEQ ID NO:31;
(e) a light chain CDR3 comprising SEQ ID NO:36, a heavy chain CDR1 comprising SEQ ID NO:33, a heavy chain CDR2 comprising SEQ ID NO:34, and a heavy chain CDR3 comprising SEQ ID NO:35;
(f) a light chain CDR3 comprising SEQ ID NO:40, a heavy chain CDR1 comprising SEQ ID NO:37, a heavy chain CDR2 comprising SEQ ID NO:38, and a heavy chain CDR3 comprising SEQ ID NO:39;
(g) a light chain CDR3 comprising SEQ ID NO:44, a heavy chain CDR1 comprising SEQ ID NO:41, a heavy chain CDR2 comprising SEQ ID NO:42, and a heavy chain CDR3 comprising SEQ ID NO:43;
(h) a light chain CDR3 comprising SEQ ID NO:48, a heavy chain CDR1 comprising SEQ ID NO:45, a heavy chain CDR2 comprising SEQ ID NO:46, and a heavy chain CDR3 comprising SEQ ID NO:47;
(i) a light chain CDR3 comprising SEQ ID NO:52, a heavy chain CDR1 comprising SEQ ID NO:49, a heavy chain CDR2 comprising SEQ ID NO:50, and a heavy chain CDR3 comprising SEQ ID NO:51; or
(j) a light chain CDR3 comprising SEQ ID NO:56, a heavy chain CDR1 comprising SEQ ID NO:53, a heavy chain CDR2 comprising SEQ ID NO:54, and a heavy chain CDR3 comprising SEQ ID NO:55.

21. The method of claim 19, wherein the antibody or antibody fragment comprises light chain CDRs of SEQ ID NO:11, SEQ ID NO:13 and SEQ ID NO:20, and heavy chain CDRs of SEQ ID NO:17, SEQ ID NO:18, and SEQ ID NO:19.

22. The method of claim 19, wherein the antibody or antibody fragment comprises a heavy chain variable domain (VH) and a light chain variable domain (VL), and the VH comprises an amino acid sequence having at least 95% sequence identity to SEQ ID NO:57, and the VL comprises an amino acid sequence having at least 95% sequence identity to SEQ ID NO:58.

23. The method of claim 19, wherein the antibody or antibody fragment comprises a heavy chain variable domain (VH) and a light chain variable domain (VL), and the VH comprises the amino acid sequence of SEQ ID NO:57, and the VL comprises the amino acid sequence of SEQ ID NO:58.

24. The method of claim 19, wherein the diagnostic radioisotope is selected from the group consisting of ¹⁸F, 44Sc ⁶⁴Cu, ⁶⁸Ga, ⁸⁹Zr, and ¹²⁴I.

25. The method of claim 19, wherein the diagnostic radioisotope is ⁸⁹Zr.

26. The method of claim 19, wherein the radionuclide imagining comprises positron emission tomography (PET) or single-photon emission computed tomography (SPECT).

27. The method of claim 19, wherein cells of the prostate cancer are prostate-specific membrane antigen (PSMA) negative.

28. The method of claim 19, wherein the prostate cancer is castration-resistant prostate cancer (CRPC), optionally wherein the prostate cancer is metastatic castration-resistant prostate cancer (mCRPC).

29. The method of claim 19, wherein the prostate cancer is an adenocarcinoma subtype or a neuroendocrine subtype.

30. The method of claim 19, further comprising determining the subject has a CUB domain-containing protein 1 (CDCP1)-positive prostate cancer, and treating the CDCP1-positive prostate cancer by a method comprising:
administering to the subject a therapeutic radioisotope-labeled antibody or antibody fragment that specifically binds CDCP1 ectodomain to treat the prostate cancer, wherein the therapeutic radioisotope is an alpha-emitter and/or a beta-minus-emitter.


SEQUENCE LISTING (80 sequences)

SEQ ID NO: 1 - protein, 246 aa, synthetic construct
EISEVQLVESGGGLVQPGGSLRLSCAASGFNFSSSSIHWVRQAPGKGLEWVASISSSYGYTYYADSVKGRFTISADTSKNTAYLQMNSLRAEDTAVYYCARTVRGSKKPYFSGWAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTGGSHHHHHH

SEQ ID NO: 2 - protein, 31 aa, synthetic construct
EISEVQLVESGGGLVQPGGSLRLSCAASGFN

SEQ ID NO: 3 - protein, 6 aa, synthetic construct
FSSSSI

SEQ ID NO: 4 - protein, 15 aa, synthetic construct
HWVRQAPGKGLEWVA

SEQ ID NO: 5 - protein, 10 aa, synthetic construct
SISSSYGYTY

SEQ ID NO: 6 - protein, 39 aa, synthetic construct
YADSVKGRFTISADTSKNTAYLQMNSLRAEDTAVYYCAR

SEQ ID NO: 7 - protein, 15 aa, synthetic construct
TVRGSKKPYFSGWAM

SEQ ID NO: 8 - protein, 13 aa, synthetic construct
DYWGQGTLVTVSS

SEQ ID NO: 9 - protein, 226 aa, synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQSSYSLITFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGECGGSDYKDDDDK

SEQ ID NO: 10 - protein, 24 aa, synthetic construct
SDIQMTQSPSSLSASVGDRVTITC

SEQ ID NO: 11 - protein, 11 aa, synthetic construct
RASQSVSSAVA

SEQ ID NO: 12 - protein, 15 aa, synthetic construct
WYQQKPGKAPKLLIY

SEQ ID NO: 13 - protein, 7 aa, synthetic construct
SASSLYS

SEQ ID NO: 14 - protein, 34 aa, synthetic construct
GVPSRFSGSRSGTDFTLTISSLQPEDFATYYCQQ

SEQ ID NO: 15 - protein, 6 aa, synthetic construct
SSYSLI

SEQ ID NO: 16 - protein, 11 aa, synthetic construct
TFGQGTKVEIK

SEQ ID NO: 17 - protein, 6 aa, synthetic construct
LSYYYI

SEQ ID NO: 18 - protein, 10 aa, synthetic construct
SIYSSSSYTS

SEQ ID NO: 19 - protein, 5 aa, synthetic construct
AYYGF

SEQ ID NO: 20 - protein, 7 aa, synthetic construct
SYYYYPI

SEQ ID NO: 21 - protein, 6 aa, synthetic construct
ISYYSM

SEQ ID NO: 22 - protein, 10 aa, synthetic construct
SISPYSGYTS

SEQ ID NO: 23 - protein, 5 aa, synthetic construct
GYYAL

SEQ ID NO: 24 - protein, 7 aa, synthetic construct
YYYFYPF

SEQ ID NO: 25 - protein, 6 aa, synthetic construct
ISYYYM

SEQ ID NO: 26 - protein, 10 aa, synthetic construct
SIYSSYGYTS

SEQ ID NO: 27 - protein, 5 aa, synthetic construct
VYYGF

SEQ ID NO: 28 - protein, 7 aa, synthetic construct
SYYVYPI

SEQ ID NO: 29 - protein, 6 aa, synthetic construct
ISYYYI

SEQ ID NO: 30 - protein, 10 aa, synthetic construct
SIYPYYGSTY

SEQ ID NO: 31 - protein, 5 aa, synthetic construct
AYYGF

SEQ ID NO: 32 - protein, 7 aa, synthetic construct
SYWSFPI

SEQ ID NO: 33 - protein, 6 aa, synthetic construct
LYYSYM

SEQ ID NO: 34 - protein, 10 aa, synthetic construct
YISPYSGSTY

SEQ ID NO: 35 - protein, 6 aa, synthetic construct
YSYSAL

SEQ ID NO: 36 - protein, 8 aa, synthetic construct
SSWHYHLF

SEQ ID NO: 37 - protein, 6 aa, synthetic construct
IYSYYI

SEQ ID NO: 38 - protein, 10 aa, synthetic construct
SIYPYYGYTS

SEQ ID NO: 39 - protein, 5 aa, synthetic construct
AYYGM

SEQ ID NO: 40 - protein, 7 aa, synthetic construct
SYFYWPI

SEQ ID NO: 41 - protein, 6 aa, synthetic construct
ISSYYM

SEQ ID NO: 42 - protein, 10 aa, synthetic construct
SIYPYSGYTY

SEQ ID NO: 43 - protein, 5 aa, synthetic construct
AYYAM

SEQ ID NO: 44 - protein, 7 aa, synthetic construct
SYYVYPI

SEQ ID NO: 45 - protein, 6 aa, synthetic construct
LYSYYI

SEQ ID NO: 46 - protein, 10 aa, synthetic construct
SIYPYYSSTS

SEQ ID NO: 47 - protein, 5 aa, synthetic construct
YYYAM

SEQ ID NO: 48 - protein, 9 aa, synthetic construct
GYAGSWHPI

SEQ ID NO: 49 - protein, 6 aa, synthetic construct
IYSYSM

SEQ ID NO: 50 - protein, 10 aa, synthetic construct
SISPYYSYTS

SEQ ID NO: 51 - protein, 5 aa, synthetic construct
AYYAL

SEQ ID NO: 52 - protein, 7 aa, synthetic construct
SYWYYPI

SEQ ID NO: 53 - protein, 6 aa, synthetic construct
ISYYYM

SEQ ID NO: 54 - protein, 10 aa, synthetic construct
SIYSSSSYTS

SEQ ID NO: 55 - protein, 5 aa, synthetic construct
SYYAM

SEQ ID NO: 56 - protein, 7 aa, synthetic construct
SYYVYPI

SEQ ID NO: 57 - protein, 116 aa, synthetic construct
EVQLVESGGGLVQPGGSLRLSCAASGFNLSYYYIHWVRQAPGKGLEWVASIYSSSSYTSYADSVKGRFTISADTSKNTAYLQMNSLRAEDTAVYYCARAYYGFDYWGQGTLVTVSS

SEQ ID NO: 58 - protein, 108 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQSYYYYPITFGQGTKVEIK

SEQ ID NO: 59 - protein, 116 aa, synthetic construct
EVQLVESGGGLVQPGGSLRLSCAASGFNISYYSMHWVRQAPGKGLEWVASISPYSGYTSYADSVKGRFTISADTSKNTAYLQMNSLRAEDTAVYYCARGYYALDYWGQGTLVTVSS

SEQ ID NO: 60 - protein, 108 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQYYYFYPFTFGQGTKVEIK

SEQ ID NO: 61 - protein, 116 aa, synthetic construct
EVQLVESGGGLVQPGGSLRLSCAASGFNISYYYMHWVRQAPGKGLEWVASIYSSYGYTSYADSVKGRFTISADTSKNTAYLQMNSLRAEDTAVYYCARVYYGFDYWGQGTLVTVSS

SEQ ID NO: 62 - protein, 108 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQSYYVYPITFGQGTKVEIK

SEQ ID NO: 63 - protein, 116 aa, synthetic construct
EVQLVESGGGLVQPGGSLRLSCAASGFNISYYYIHWVRQAPGKGLEWVASIYPYYGSTYYADSVKGRFTISADTSKNTAYLQMNSLRAEDTAVYYCARAYYGFDYWGQGTLVTVSS

SEQ ID NO: 64 - protein, 108 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQSYWSFPITFGQGTKVEIK

SEQ ID NO: 65 - protein, 117 aa, synthetic construct
EVQLVESGGGLVQPGGSLRLSCAASGFNLYYSYMHWVRQAPGKGLEWVAYISPYSGSTYYADSVKGRFTISADTSKNTAYLQMNSLRAEDTAVYYCARYSYSALDYWGQGTLVTVSS

SEQ ID NO: 66 - protein, 109 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQSSWHYHLFTFGQGTKVEIK

SEQ ID NO: 67 - protein, 116 aa, synthetic construct
EVQLVESGGGLVQPGGSLRLSCAASGFNIYSYYIHWVRQAPGKGLEWVASIYPYYGYTSYADSVKGRFTISADTSKNTAYLQMNSLRAEDTAVYYCARAYYGMDYWGQGTLVTVSS

SEQ ID NO: 68 - protein, 108 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQSYFYWPITFGQGTKVEIK

SEQ ID NO: 69 - protein, 116 aa, synthetic construct
EVQLVESGGGLVQPGGSLRLSCAASGFNISSYYMHWVRQAPGKGLEWVASIYPYSGYTYYADSVKGRFTISADTSKNTAYLQMNSLRAEDTAVYYCARAYYAMDYWGQGTLVTVSS

SEQ ID NO: 70 - protein, 108 aa, synthetic construct; identical to SEQ ID NO: 62

SEQ ID NO: 71 - protein, 116 aa, synthetic construct
EVQLVESGGGLVQPGGSLRLSCAASGFNLYSYYIHWVRQAPGKGLEWVASIYPYYSSTSYADSVKGRFTISADTSKNTAYLQMNSLRAEDTAVYYCARYYYAMDYWGQGTLVTVSS

SEQ ID NO: 72 - protein, 110 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQGYAGSWHPITFGQGTKVEIK

SEQ ID NO: 73 - protein, 116 aa, synthetic construct
EVQLVESGGGLVQPGGSLRLSCAASGFNIYSYSMHWVRQAPGKGLEWVASISPYYSYTSYADSVKGRFTISADTSKNTAYLQMNSLRAEDTAVYYCARAYYALDYWGQGTLVTVSS

SEQ ID NO: 74 - protein, 108 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQSYWYYPITFGQGTKVEIK

SEQ ID NO: 75 - protein, 116 aa, synthetic construct
EVQLVESGGGLVQPGGSLRLSCAASGFNISYYYMHWVRQAPGKGLEWVASIYSSSSYTSYADSVKGRFTISADTSKNTAYLQMNSLRAEDTAVYYCARSYYAMDYWGQGTLVTVSS

SEQ ID NO: 76 - protein, 108 aa, synthetic construct; identical to SEQ ID NO: 62

SEQ ID NO: 77 - protein, 126 aa, synthetic construct
EVQLVESGGGLVQPGGSLRLSCAASGFNFSSSSIHWVRQAPGKGLEWVASISSSYGYTYYADSVKGRFTISADTSKNTAYLQMNSLRAEDTAVYYCARTVRGSKKPYFSGWAMDYWGQGTLVTVSS

SEQ ID NO: 78 - protein, 107 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQSSYSLITFGQGTKVEIK

SEQ ID NO: 79 - protein, 14 aa, synthetic construct
DKTHTGGSHHHHHH

SEQ ID NO: 80 - protein, 11 aa, synthetic construct
GGSDYKDDDDK